(19) United States
(12) Patent Application Publication
(10) Pub. No.: US 2001/0012854 A1
(43) Pub. Date: Aug. 9, 2001

(54) MULTICATALYTIC PROTEASE INHIBITORS FOR USE AS ANTI-TUMOR AGENTS

(76) Inventors: ROBERT SIMAN, WILMINGTON, DE (US); JITESH P. JANI, WATERFORD, CT (US); RONALD H. GOLDFARB, ARLINGTON, TX (US); QING PING DOU, TAMPA, FL (US)

Correspondence Address: MICHAEL P STRAHER, WOODCOCK WASHBURN KURTZ, MACKIEWICZ & NORRIS, ONE LIBERTY PLACE - 46TH FLOOR, PHILADELPHIA, PA, 19103

(21) Appl. No.: 09/211,951

(22) Filed: Dec. 15, 1998

Related U.S. Application Data
(60) Provisional application No. 60/069,804, filed on Dec. 16, 1997.

(51) Int. Cl. A61K 38/00

(52) U.S. Cl. 514/463; 514/19; 514/183; 514/452; 514/519; 514/590; 514/616; 564/123; 554/57

(57) ABSTRACT

The present invention relates to methods for the use of specified inhibitors of multicatalytic protease (MCP) for use as inducers of programmed cell death (i.e., apoptosis) in tumor cells, and more particularly as anti-tumor agents. The present invention provides methods for inducing apoptosis in transformed cells, inhibiting proliferation of transformed cells, and inhibiting the growth of tumors using the MCP inhibitors.

### CROSS REFERENCE TO RELATED APPLICATIONS

This application claims priority benefit of U.S. provisional application Ser. No. 60/069,804, filed Dec. 16, 1997, the content of which is incorporated herein by reference in its entirety.

### FIELD OF THE INVENTION

This invention relates to specified inhibitors of multicatalytic protease (MCP) as disclosed in U.S. Pat. Nos. 5,614,649 and 5,550,262 for use as inducers of programmed cell death (i.e., apoptosis) in tumor cells, and more particularly as anti-tumor agents.

### BACKGROUND OF THE INVENTION

### I. Apoptosis In Cancerous Conditions

Apoptosis is an active process, e.g., of programmed cell death that is conserved throughout evolution from worm to humans (Jacobson, M. J., et al. Cell 88, 347-354, 1997). Apoptosis occurs in two physiological stages, commitment and execution. The apoptotic execution is initiated by activation of specific proteases of the caspase family, which exhibit an unusual substrate specificity, i.e., cleavage after aspartic acid (“Asp”) residues (Martin, S. J. and Green, D. R. Cell, 82:349-352, 1995). To date, at least ten homologs of caspases have been identified and cloned (Alnemri, E. S., et al. Cell 87, 171, 1996). Activation of caspases leads to apoptosis, most likely via the proteolytic cleavage of important cellular proteins. It has been reported that a number of proteins, including poly(ADP)-ribose polymerase (“PARP”; Lazebnik, Y. A., et al. Nature, 371:346-347, 1994), actin (Kayalar, C., et al. PNAS USA, 93:2234-2238, 1996), sterol regulatory binding proteins (Wang, X., et al. EMBO J., 15:1012-1020, 1996) and DNA-dependent protein kinase (Song, Q., et al. EMBO J., 15:3238-3246, 1996), are cleaved after Asp residues during apoptosis, indicating that a caspase is the cleavage enzyme. It has been reported (An, B., and Dou, Q. P., Cancer Res. 56:438-442, 1996) that when HL-60 or U937 cells were treated with anticancer agents, retinoblastoma (RB) protein, an important cell cycle regulator and tumor suppressor (Weinberg, R. A. Cell, 81:323-330, 1995), is proteolytically cleaved into two major fragments, referred to as “p68” and “p48.” Both the RB interior cleavage and apoptosis were reported to be inhibitable by different caspase inhibitors, such as YVAD-CMK (An, B., and Dou, Q. P. Cancer Res., supra), the Bcl-2 oncoprotein, or the cowpox virus CrmA protein (Dou, Q. P., et al. J Cell Biochem., 64:586-594, 1997). Subsequently, other groups have reported that during apoptosis, RB was also cleaved from its C-terminus by a caspase 3-like protease (Janicke, R. U., et al. EMBO J 15, 6969-6978, 1996; Chen, W., et al. Oncogene 14, 1243-1248, 1997; Tan, X., et al. J. Biol. Chem. 272, 9613-9616, 1997). It has also been reported that RB became dephosphorylated prior to the endonucleosomal fragmentation of DNA (Dou, Q. P., et al. PNAS USA, 92:9019-9023, 1995; Wang, H., et al. Oncogene, 13:373-379, 1996; and Morana, et al. J. Biol Chem. 271:18263-18271, 1996).

Activation of the cellular apoptotic program is a current strategy for treatment of human cancers. It has been demonstrated that X-irradiation and standard chemotherapeutic drugs kill some tumor cells through induction of apoptosis (Fisher, D. E. Cell 78, 539-542, 1994). Unfortunately, the majority of human cancers at present are resistant to these therapies (Harrison, D. J. J. Patho. 175, 7-12, 1995). Although the molecular mechanisms for development of such multidrug resistance in human cancers are unclear, it has been suggested that overexpression of Bcl-2 (Reed, J. C. J. Cell. Bio. 124, 1-6, 1994), inactivation of the tumor suppressor protein “p53 ” (Milner, J. Nature Medicine 1, 789-880, 1995), or activation of a survival program through the transcriptional regulator NF-κB (Beg, A. A. and Baltimore, D., Science 274: 782-784, 1996; Wang et.al., Science 274: 784-787, 1996), are involved in this process.

### II. Multicatalytic Protease (MCP)

Eukaryotic cells constantly degrade and replace cellular protein. This permits the cell to selectively and rapidly remove proteins and peptides having abnormal conformations, to exert control over metabolic pathways by adjusting levels of regulatory peptides, and to provide amino acids for energy when necessary, as in starvation (Goldberg, A. L. & St. John, A. C. Annu. Rev. Biochem. 45:747-803, 1976). The cellular mechanisms of mammals allow for multiple pathways for protein breakdown. Some of these pathways appear to require energy input in the form of adenosine triphosphate (“ATP”) (Goldberg, A. L. & St. John, A. C. supra).

Multicatalytic Protease (MCP, also typically referred to as “multicatalytic proteinase,” “proteasome,” “multicatalytic proteinase complex,” “multicatalytic endopeptidase complex,” “20S proteasome” and “ingensin”) is a large molecular weight (700 kD) eukaryotic non-lysosomal proteinase complex which plays a role in at least two cellular pathways for the breakdown of protein to peptides and amino acids (Orlowski, M. Biochemistry 29(45) 10289-10297, 1990). The complex has at least five different types of hydrolytic activities: (1) a trypsin-like activity wherein peptide bonds are cleaved at the carboxyl side of basic amino acids; (2) a chymotrypsin-like activity wherein peptide bonds are cleaved at the carboxyl side of hydrophobic amino acids; (3) an activity wherein peptide bonds are cleaved at the carboxyl side of glutamic acid; (4) a branched-chain amino acid preferring activity; and (5) a small neutral amino acid preferring activity (Rivett, A. J. J. Biol. Chem. 264:21 12215-12219, 1989; and Orlowski, supra).

One route of protein hydrolysis which involves MCP also involves the polypeptide “ubiquitin” (Hershko, A. & Ciechanover, A. Annu. Rev. Biochem. 51:335-364, 1982). This route, which requires MCP, ATP and ubiquitin, appears responsible for the degradation of highly abnormal proteins, certain short-lived normal proteins and the bulk of proteins in growing fibroblasts and maturing reticuloytes. (Driscoll, J. and Goldberg, A. L. PNAS USA 86:787-791, 1989). Proteins to be degraded by this pathway are covalently bound to ubiquitin via their lysine amino groups in an ATP-dependent manner. The ubiquitin-conjugated proteins are then degraded to small peptides by an ATP-dependent protease complex, the 26S proteasome, which contains MCP as its proteolytic core (Goldberg, A. L. & Rock, K. L. Nature 357:375-379, 1992).

A second route of protein degradation which requires MCP and ATP, but which does not require ubiquitin, has also been described (Driscoll, J. & Goldberg, A. L., supra). In this process, MCP hydrolyzes proteins in an ATP-dependent manner (Goldberg, A. L. & Rock, K. L., supra). This process has been observed in skeletal muscle (Driscoll & Goldberg, supra). However, it has been suggested that in muscle, MCP functions synergistically with another protease, multipain, thus resulting in an accelerated breakdown of muscle protein (Goldberg & Rock, supra).

It has been reported that MCP functions by a proteolytic mechanism wherein the active site nucleophile is the hydroxyl group of the N-terminal threonine residue. Thus, MCP is the first known example of a threonine protease (Seemuller et al., Science 268 579-582, 1995; Goldberg, A. L, Science 268 522-523, 1995).

Recent studies have suggested that the MCP system is involved in the regulation of apoptosis, although this postulated role remains controversial. It has been found that some MCP inhibitors, such as tripeptide aldehydes (e.g., LLnL or LLnV) or lactacystin (a microbial metabolite), block the process of programmed cell death in thymocytes (Grimm, L. M., et al. EMBO J. 15, 3835-3844, 1996) and neurons (Sadoul, R., et al. EMBO J. 15, 3845-3852, 1996). In contrast, the same or similar MCP inhibitors have been found to induce apoptosis in human leukemia (Imajoh-Ohmi, et al. Biochem. Biophy. Res. Commu. 217, 1070-1077, 1995; Shinohara, K., et al. Biochem. J. 317, 385-388, 1996; and Drexler, H. C. A. PNAS USA 94, 855-860, 1997) and other proliferating cell lines (Lopes, U. G., et al. J. Biol. Chem. 272, 12893-1896, 1997).

### III. MCP Inhibitors

The specified inhibitors of MCP which are the subject of this invention are disclosed in U.S. Pat. Nos. 5,614,649 and 5,550,262, both of which are assigned to Cephalon, Inc. These patent documents are incorporated herein by reference in their entirety.

### SUMMARY OF THE INVENTION

The present invention is directed to the use of MCP inhibitors as inducers of programmed cell death (i.e., apoptosis), and as anti-tumor agents.

In some preferred embodiments, the present invention provides methods for causing the death of transformed cells comprising contacting said cells with a compound of the invention.

In further preferred embodiments, the present invention provides methods for treating a patient having a disease, said disease being characterized by the presence of transformed cells, comprising contacting said cells with a compound of the invention.

In still further preferred embodiments, methods are provided for inducing apoptosis in cells comprising contacting said cells with a compound of the invention.

Also provided in accordance with the present invention are methods for inhibiting proliferation of transformed cells comprising contacting said cells with a compound of the invention, and methods for inhibiting the growth of a tumor comprising contacting said tumor with a compound of then invention. In some preferred embodiments, the tumor is a solid tumor.

In some preferred embodiments of the methods of the invention, the compound is administered to a mammal, preferably a human.

In some preferred embodiments of the methods of the invention, the transformed cells are breast cancer cells, prostate cancer cells, tongue cancer cells, brain cancer cells, lung cancer cells, pancreatic cancer cells, ovarian cancer cells, or skin cancer cells.

In some further preferred embodiments, the transformed cells overproduce Bcl2 protein, and/or lack p53 protein.

The MCP inhibitors of the invention are disclosed in U.S. Pat. Nos. 5,550,262 and 5,614,649, the disclosures of each of which are incorporated herein by reference in their entirety. These compounds are represented by the formula:

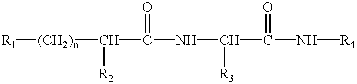

Constituent members are defined infra, as well as preferred constituent members for preferred anti-tumor agents. These compounds are useful inducers of apoptosis applicable in a variety of tumor cell types, and in particular solid tumors resistant to treatment with currently-approved chemotherapeutic agents.

These and other features of the invention will be set forth in expanded form as the disclosure continues.

### BRIEF DESCRIPTION OF THE DRAWINGS

FIGS. 1 and 1A show the effects of disclosed MCP inhibitors as apoptotic inducers in human leukemia cells.

FIGS. 2 and 2A compare the apoptosis-inducing potency of disclosed MCP inhibitors.

FIG. 3 is a reproduction of a photograph showing that overexpression of the Bcl-2 oncoprotein fails to inhibit apoptotic nuclear changes induced by Compound A.

FIG. 4 is a reproduction of a photograph showing that overexpression of the Bcl-2 oncoprotein fails to inhibit cleavage of PARP and production of p112-115/RB induced by Compound A.

FIG. 5 is a reproduction of a photograph showing induction of detachment and apoptosis by Compound A, but not by either etoposide or cisplatin, in several human cancer cell lines.

FIG. 6 is a reproduction of a photograph showing that Compound A selectively induces apoptotic nuclear changes in SV40-transformed, but not the parental normal, human fibroblasts.

FIG. 7 shows that Compound A selectively induces PARP cleavage in SV40-transformed, but not the parental normal, human fibroblasts.

FIG. 8 is a graphic representation showing in vivo anti-tumor activity of Compounds D and E.

FIG. 9 is a graphic representation showing in vivo inhibition of lung carcinoma tissue growth by Compounds I and J.

FIG. 10 is a graphic representation showing in vivo inhibition of rat prostatic carcinoma by compound I.

### DETAILED DESCRIPTION

MCP inhibitors useful in the induction of apoptosis for use as anti-tumor agents in accordance with the invention are represented by the formula:

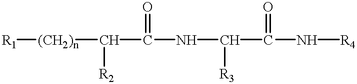

wherein:

R₁ is selected from the group consisting of —C≡N, —C(═O)OR₉, phthalimido, —NH— SO₂R₉, and —NH—J;

R₂ is selected from the group consisting of H, hydroxyl, alkyl having from one to ten carbons, and cycloalkyl having from three to seven carbons;

R₃ is selected from the group consisting of —(CH₂)ₘ—NH—C(═N—R₅)—NH₂, —R₆—NO₂, —R₆—J, and —R₆—C≡N;

R₄ is —CH(CH₂—R₇)—Q;

Q is selected from the group consisting of —CH—R₈, —C(═O)CH₃, —C(═O)CH₂Cl, —C(═O)CH₂Br, —C(═O)CH₂F, —C(═O)CHF₂, —C(═O)CF₃, —C(═O)C(═O)R₇, —C(═O)C(═O)NH—R₇, —C(═O)CO₂—R₇, —C(═O)CO₂H, —B(OH)₂,

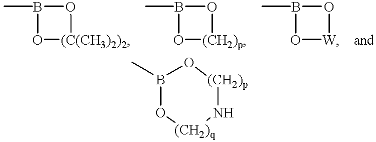

where p and q, independently, are 2 or 3;

W is cycloalkyl;

R₅ is selected from the group consisting of —NO₂, —C═N, and —J;

R₆ is —(CH₂)ₘ—NH—C(═NH)—NH—;

R₇ is selected from the group consisting of phenyl, and alkyl having from one to eight carbons, said alkyl group being optionally substituted with one or more halogen atoms, aryl, or heteroaryl groups;

R₈ is selected from the group consisting of ═O, ═N—NHC(═O)—NH₂, ═N—OH, ═N—OCH₃, ═N—O—CH₂—C₆H₅, ═NNH—C(═S)—NH₂ and ═N—NH—J;

R₉ is selected from the group consisting of hydrogen and alkyl having from one to six carbons, said alkyl group being optionally substituted with one or more halogen atoms, aryl or heteroaryl groups;

J is a protecting group;

n is an integer from 3 to 10; and

m is an integer from 2 to 5.

In some preferred embodiments R₁ is —C≡N, —C(═O)OCH₃, phthalimido or —NH— SO₂CF₃, and in other preferred embodiments R₂ is H or cyclopentyl.

R₃ is preferably —(CH₂)₃—NH—C(═N—R₅)—NH₂.

Q is preferably —CH—R₈, —B(OH)₂, —C(═O)C(═O)NH—R₇, or has the structure:

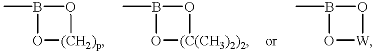

where W is pinane.

R₅ is preferably —NO₂, —C≡N, —PMC, —MTR, —MTS, or Tos.

R₇ is preferably —CH(CH₃)₂, —(CH₂)₂—CH₃, —CH₂—CH₃, or —C₆H₅.

R₈ is preferably ═O, ═N—OH, ═N—O—CH₂—C₆H₅, ═NNH—C(═O)—NH₂ or ═NNH—C(═S)—NH₂.

In some preferred embodiments R₁ is —C(═O)OCH₃, phthalimido or —NH—SO₂CF₃; R₂ is cyclopentyl; R₃ is —(CH₂)₃—NH—C(═N—NO₂)—NH₂; R₇ is —CH(CH₃)₂; and R₈ is ═O.

In other preferred embodiments R₁ is —C≡N; R₂ is cyclopentyl; R₃ is —(CH₂)₃—NH— C(═N—NO₂)—NH₂ or —(CH₂)₃—NH—C(═N—J)—NH₂; R₇ is —CH(CH₃)₂; and R₈ is ═O.

In further preferred embodiments R₁ is C≡N; R₂ is cyclopentyl; R₃ is —(CH₂)₃—NH— C(═N—NO₂)—NH₂ or —(CH₂)₃—NH—C(═N—J)—NH₂; R₇ is —CH(CH₃)₂; Q is —CH—R₈; and R₈ is ═N—NHC(═O)—NH₂, ═N—OH, ═N—OCH₃, or ═N—O—CH₂—C₆H₅.

In some particularly preferred embodiments, R₁, R₂, R₃ and R₄ are selected from the group of substituents shown for the compounds in Table 1. In some especially preferred embodiments, R₁, R₂, R₃ and R₄ are selected to form compounds A-J shown in Table 1, infra.

As used herein, the term “alkyl” is meant to include straight-chain, branched and cyclic hydrocarbons such as ethyl, isopropyl and cyclopentyl groups. Substituted alkyl groups are alkyl groups for which one or more hydrogen atoms have been replaced by halogen, other hydrocarbon groups (for example, a phenyl group), a heteroaryl group, or a group in which one or more carbon atoms are interrupted by oxygen atoms. Preferred alkyl groups have 1 to about 8 carbon atoms. As used herein, the term “halogen” has its usual meaning and includes fluorine, chlorine, bromine and iodine, with fluorine being a preferred halogen. The term “Arg” as used in the present invention has its normal meaning as the abbreviation for the amino acid “arginine.”

It will be appreciated that each of the structural formulas described herein are intended to include their corresponding tautomeric forms, such as are shown below:

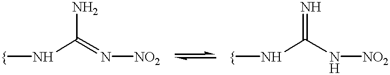

Embodiments of the MCP inhibitors may contain protecting groups. As used herein, the phrase “protecting groups” is to be accorded a broad interpretation. Protecting groups are known per se as chemical functional groups that can be selectively appended to and removed from functionalities, such as hydroxyl groups, amino groups and carboxyl groups. These groups are present in a chemical compound to render such functionality inert to chemical reaction conditions to which the compound is exposed. Any of a variety of protecting groups may be employed with the present invention. One such protecting group is the phthalimido group. Other preferred protecting groups according to the invention have the following formulas:

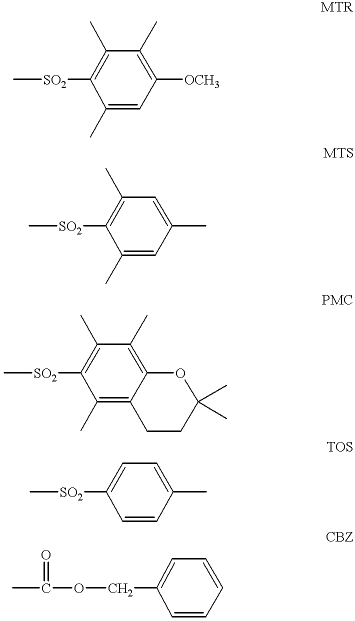

Further representative protecting groups suitable for practice in the invention may be found in Greene, T. W. and Wuts, P. G. M., “Protecting Groups in Organic Synthesis” 2d. Ed., Wiley & Sons, 1991.

As previously indicated, MCP inhibitors may either induce or block apoptosis depending upon the cell type. The MCP inhibitors disclosed herein kill tumor cells by apoptosis, even for tumor lines resistant to chemotherapeutic agents. The usefulness of such compounds can be applied to both research and therapeutic settings. Methodologies for inhibiting the activity of MCP by contacting the MCP with a compound of the invention include providing the compound to a mammal, including a human, as a medicament or pharmaceutical agent.

As used herein, the term “contacting” means directly or indirectly causing placement together of moieties to be contacted, such that the moieties come into physical contact with each other. Contacting thus includes physical acts such as placing the moieties together in a container, or administering moieties to a patient. Thus, for example, administering a compound of the invention to a human patient evidencing a disease or disorder associated with abnormal cell proliferation, or involving the presence of transformed cells, falls within the scope of the definition of term “contacting.”

In preferred embodiments, pharmaceutical compositions according to the invention are administered to patients suffering from a disorder, i.e., an abnormal physical condition, a disease or pathophysiological condition associated with normal, abnormal and/or aberrant activities of MCP, e.g., interference with the regulation of apoptosis. The disorders for which the compositions of the invention are administered are preferably those which directly or indirectly inhibit or abnormally interferes with apoptosis, and in particular, those situations where such inhibition or abnormal interference leads to or results in cancerous conditions.

Some Diseases in which cell elimination by induction of apoptosis is desirable include various cancers, including, for example, melanoma, prostate, pancreas, ovary, mammary, tongue, and lung cancers. Tumors treatable with the methods of the present invention include and are not limited to melanoma, prostate, pancreas, ovary, mammary, tongue, lungs, and smooth muscle tumors; as well as cells from glioblastoma, bone marrow stem cells, hematopoietic cells, osteoblasts, epithelial cells, and fibroblasts. Those having ordinary skill in the art can readily identify individuals who are suspected of suffering from such diseases, conditions and disorders using standard diagnostic techniques.

Cells can be treated in vivo or ex vivo in accordance with the methods of the invention. For in vivo treatment, cells of an animal, preferably a mammal and most preferably a human, are contacted with a compound of the invention by any of a variety of modes of administration as are known in the art. Thus, in accordance with the methods of the invention, compounds of the invention may be administered by any means that enables the active agent to reach the agent's site of action in the body of a mammal.

In the context of the invention, “administering” means introduction of the pharmaceutical composition into a patient. Preferred methods of administration include intravenous, subcutaneous and intramuscular administration. Preferably, the MCP inhibitor will be administered as a pharmaceutical composition comprising the MCP inhibitor in combination with a pharmaceutically acceptable carrier, such as physiological saline. Other suitable carriers can be found in Remington's Pharmaceutical Sciences (Mack Pub. Co., Easton, PA, 1980).

The concentrations of the compounds described herein in a pharmaceutical composition will vary depending upon a number of factors, including the dosage of the drug to be administered, the chemical characteristics (e.g., hydrophobicity) of the compounds employed, and the route of administration. In general terms, the compounds of this invention may be provided in an aqueous physiological buffer solution containing about 0.1 to 10% w/v of the MCP inhibitor for parenteral administration. Typical dose ranges are from about 1 μg/kg to about 1 g/kg of body weight per day; a preferred dose range is from about 0.01 mg/kg to 100 mg/kg of body weight per day. The preferred dosage of drug to be administered is likely to depend on such variables as the type and extent of progression of the disease or disorder, the overall health status of the particular patient, the relative biological efficacy of the compound selected, and formulation of the MCP inhibitor excipient, and its route of administration. As used herein the term “patient” denotes any type of vertebrate. Preferably, the patient is a human.

As is demonstrated by the following examples, MCP inhibitors as disclosed herein are potent inducers of apoptosis in a variety of tumor cells, thus providing utility for such compounds as anti-tumor agents. Importantly, the data disclosed herein supports the conclusion that preferred Compound A has superior apoptosis-inducing potency than either etoposide (VP-16) or cisplatin, two currently approved chemotherapeutic agents.

The invention is further illustrated by way of the following examples. These examples are intended to elucidate the invention. The examples are not intended to limit the scope of any claims appended hereto. The structures of specified compounds exemplified herein are set forth in Table 1, below.

TABLE 1
Exemplified MCP Inhibitor Structures

| Compound | Structure |
|---|---|
| A | 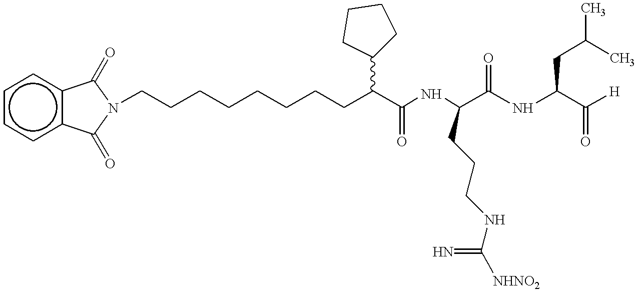 |
| B | 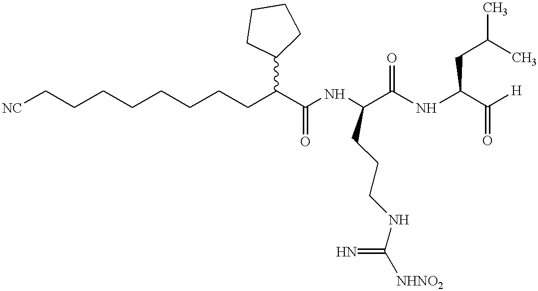 |
| C | 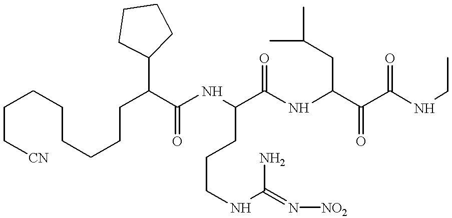 |
| D | 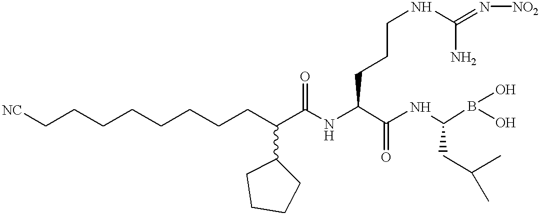 |
| E | 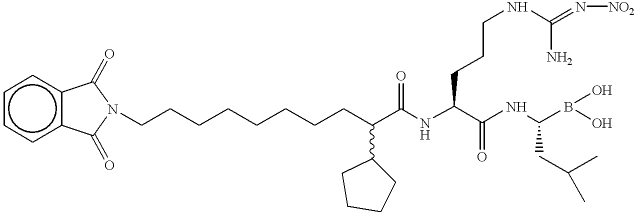 |
| F | 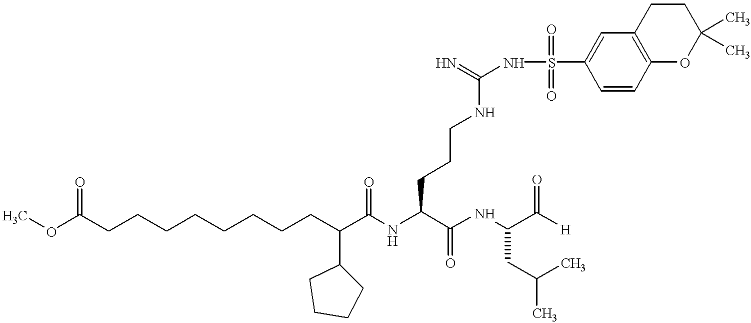 |
| G | 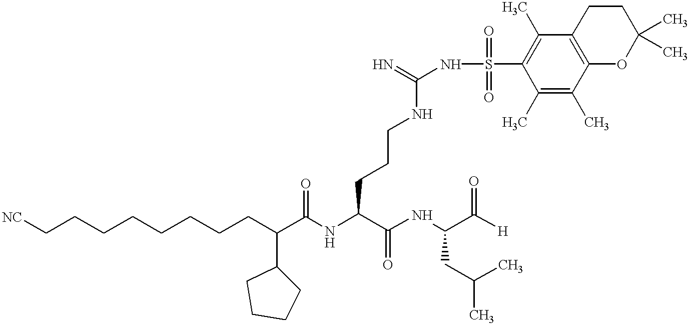 |
| H | 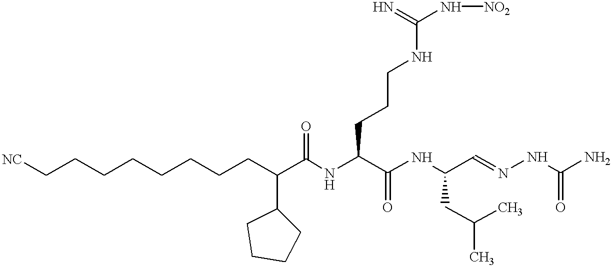 |
| I | 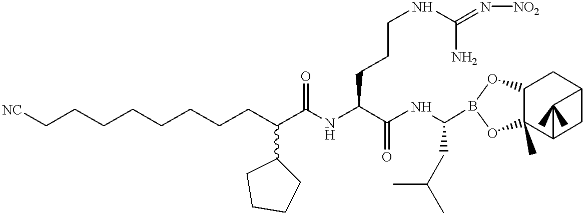 |
| J | 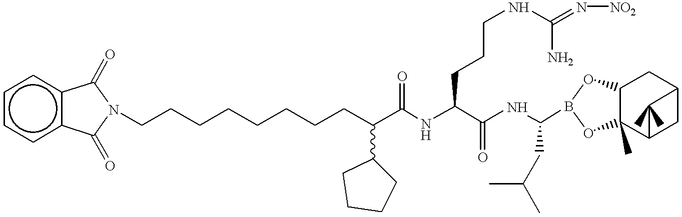 |

### EXAMPLES

### 1. In Vitro Materials and Methods

a. Materials. MCP Inhibitors were generated by Cephalon, Inc. (West Chester, Pa., USA). These compounds were synthesized in accordance with the procedures set forth in U.S. Pat. Nos. 5,614,649 and 5,550,262.

Purified mouse monoclonal antibody to: (1) human RB (G3-245) and p21 were purchased from PharMingen (San Diego, Calif.); (2) CPP32 was from Transduction Laboratories (Lexington, Ky.); and (3) human PARP (C-2-10) was from Unité de Santé at Environnement (Quebec, Canada). Purified rabbit polyclonal antibody to p27 was from Upstate Biotechnology Inc. (Lake Placid, N.Y.). Mouse monoclonal culture supernatant to human RB (XZ55) was provided by Drs. N. Dyson and E. Harlow (Massachusetts General Hospital Cancer Center, Charlestown, Mass.). Etoposide, cisplatin, propidium iodide, Hoechst 33258 and other chemicals were obtained from Sigma (St. Louis, Mo.). Acetyl-YVAD-chloromethyl ketone (YVAD-CMK) was from Bachem Bioscience Inc. (King of Prussia, Pa.).

b. Cell culture. Human Jurkat T and HL-60 cells were grown in RPMI 1640 (Life Technologies, Inc.) supplemented with 10% fetal calf serum (Sigma), 100 units/ml of penicillin, 100 μg/ml of streptomycin and 2mM L-glutamine (growth medium). Jurkat T. cells overexpressing the Bcl-2 oncoprotein or vector alone (provided by Dr. D. Johnson, University of Pittsburgh, Pa.) were grown in the same growth medium with 0.4 μg/ml G418. Human cancer cell lines of breast (MCF-7, MDA-MB-231), prostate (DU145, PC3) and osteosarcoma (U2-OS), obtained from American Type Culture Collection (ATCC. Rockville, Md.)), were also grown in the RPMI growth medium. Human oral (SCC-25; from ATCC) and brain (SNB-19; Welch, W. C., et al. In Vitro Cell. Dev. Biol., 31:610-616, 1995) cancer cell lines, and normal (WI-38) and SV40-transformed human fibroblasts (WI-38 VA-13; from ATCC) were grown in DMEM medium containing 10% fetal calf serum, penicillin, streptomycin and L-glutamine.

c. Treatment of cells with MCP inhibitor. Cells were treated with a specified MCP Inhibitor, a standard anticancer agent (etoposide or cisplatin), or DMSO (vehicle). During this process, morphological changes and cellular detachment (for attached cell lines) were monitored. At each time point, cells were harvested, and used for measurement of apoptosis and other biochemical events. In Example 1, infra, involving YVAD-CMK, Compound A was first added to Jurkat T cells. This was followed immediately by dividing the cells into multiple tissue culture flasks. YVAD-CMK was then added to an indicated concentration.

d. Flow cytometry nuclear staining and DNA fragmentation assays. DNA content analysis using flow cytometry was performed as described previously (Nicoletti, I., et al. J. Immunol. Methods 139:271-279, 1992). To assay nuclear morphology, cells were washed with PBS, fixed with 70% ethanol for 1 h, and stained with Hoechst 33258 (1 mM) for 30 min. The nuclear morphology of cells was visualized by fluorescence microscope (OLYMPUS BH2). DNA fragmentation was assayed as described previously (Grant, S., et al. Cancer Res. 52:6270-6278, 1992).

e. Whole cell extracts and western blot assay. To prepare whole cell extracts, cells were washed with PBS, and homogenized in lysis buffer (50 mM Tris, pH 8.0, 5 mM EDTA, 150 mM NaCl, 0.5% Nonidet P-40, 0.5 mM PMSF and 0.5 mM dithiothreitol). Following 30 min rocking at 4° C., the lysates were centrifuged and the supernatants were collected as whole cell extracts. The protein samples were then analyzed by sodium dodecyl sulfate-6% polyacrylamide gel electrophoresis (20-60 μg protein per lane) followed by the enhanced chemiluminescence Western blot assay using specific antibodies to CPP32, PARP, p21, p27 or RB.

f. Cell viability assay. Jurkat cells were treated with MCP inhibitors or vehicle for 30 min. at 37° C. Next, ionomycin (1 μM) and the phorbol ester PMA (10 ng/ml) were added, together with 0.2% ethanol. After 24 h, cell viability was assessed by reduction of XTT (Sigma, X4251). To each well, 50 μl of XTT (1 mg/ml) and 25 μl PMS (Sigma 9625, 5 mM) were added. After 3 h at 37° C., the OD of each well was read at 450 nm/690 nm by a plate reader.

### 2. Example 1: The Induction of Apoptosis and Activation of Caspases by the Disclosed MCP Inhibitors in Human Leukemic Cells

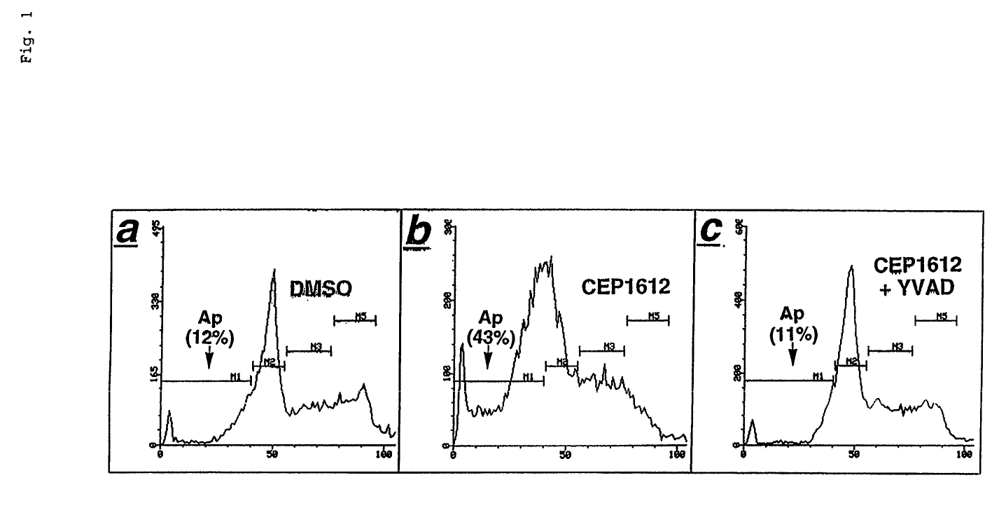

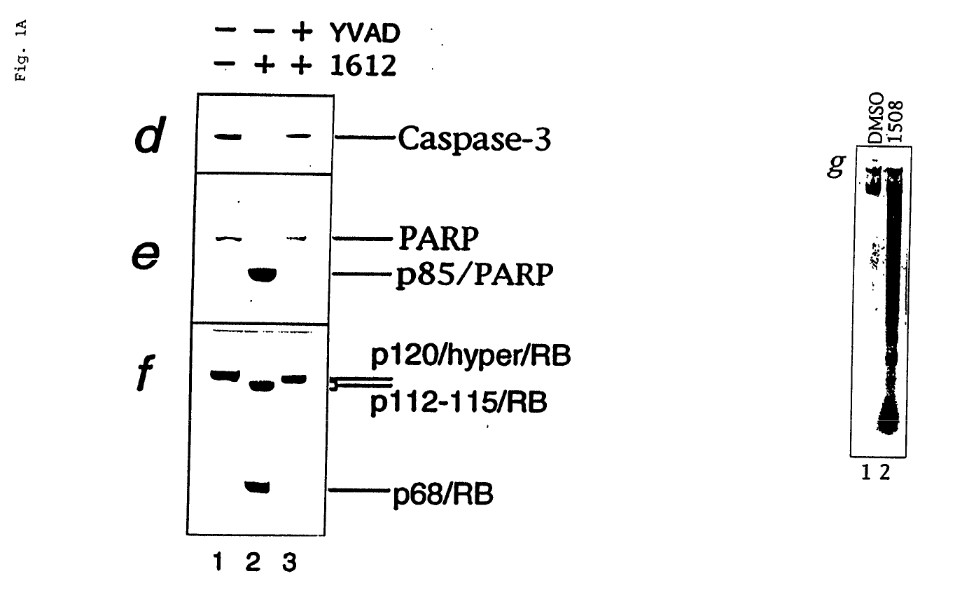

Example 1 was designed to determine if MCP is involved in the survival signaling pathway(s) and if inhibition of MCP activity induces apoptosis. Human Jurkat T cells were treated with 30 μM Compound A for 4 h. Under such conditions, apoptosis occurred as demonstrated by (a) the appearance of an apoptotic population with sub-G₁ DNA content (FIG. 1, panel b vs. a); (b) condensation and fragmentation of nuclei (comparable to FIG. 3, b vs. a); and (c) internucleosomal fragmentation of DNA (FIG. 1A, panel g). Treatment with Compound A also induced processing of caspase-3, which is required for activation of apoptosis. Such treatment also induced cleavage of PARP to a p85 fragment and cleavage of RB to a p68 fragment (FIG. 1A, d-f, lanes 2 vs. 1). The same treatment also induced the processes of RB C-terminal cleavage and dephosphorylation, as evidenced by production of the C-terminal truncated (p112) and hypophosphorylated (p115) forms of RB (FIG. 1A, f, lanes 2 vs. 1). Both the p112 and p115 forms of RB as p112-115/RB were combined and used as an apoptosis marker. All of the noted apoptotic events were also observed when human leukemia HL-60 cells were treated with Compound B. For example, exposure of HL-60 cells to Compound B induced internucleosomal fragmentation of DNA (FIG. 1A, panel g).

Because both human Jurkat T cells (Iwamoto, K. S., et al. Cancer Res. 56:3862-3865, 1996) and HL-60 cells (Danova, M., et al. Leukemia Res. 14, 417-422, 1990) contain mutant p53 genes, the foregoing data support the position that apoptosis induced by the disclosed MCP inhibitors is p53-independent.

To obtain further evidence that the Jurkat T cell apoptosis resulted from inhibition of the chymotrypsin-like activity of MCP, the cells were treated with varying concentrations of seven different compounds for 24 hours, their viability was then assessed by XTT reduction, and the cell killing potency and MCP inhibitory potency were rank-ordered. The rank-order potency for Jurkat cell killing precisely matched the order for MCP inhibition (Table 2, below).

TABLE 2
Toxicities of Compounds for the Jurkat Human T Cell
Leukemia Line and Their Potencies as MCP Inhibitors

| | Inhibition, MCP chymotrypsin- | |
|---|---|---|
| Compound | like activity (IC₅₀, nM) | Toxicity (IC₅₀, μM) |
| A | 2 | 0.5 |
| B | 6 | 1.6 |
| C | 21 | 9.7 |
| D | 5 | 0.6 |
| F | 10 | 1.7 |
| G | 20 | 5.7 |
| H | 300 | >30 |

To provide additional evidence for involvement of caspase activation in apoptosis induced by the disclosed MCP inhibitors, the inventors utilized acetyl-YVAD-chloromethyl ketone (YVAD-CMK), a tetrapeptide inhibitor that inhibits some caspase activities (Thornberry, N. A., et al. M. J. Nature, 356:768-774, 1992; and Lazebnik, Y. A., supra) and also prevents apoptosis in some cell systems (Enari, M., et al. Nature, 375:78-81, 1995; and An, B., and Dou, Q. P. supra). Addition of YVAD-CMK into Compound A- treated Jurkat T cells completely blocked: (a) production of the apoptotic peak (FIG. 1, panels c vs. b); (b) processing of caspase-3; (c) cleavage of PARP; and (d) dephosphorylation and cleavage of RB (FIG. 1A, d-f, lanes 3 vs. 2). These data support the position that a caspase is actively located upstream of these events.

### 3. Example 2: The Apoptosis-inducing Abilities of MCP Inhibitors are Proportional to their Inhibitory Activities Toward the MCP Chymotrypsin-like Activity

Inhibition of the chymotrypsin-like activity of MCP by the disclosed MCP inhibitors has been reported, but these MCP inhibitors produced relatively little inhibition on the MCP trypsin-like activity and cathepsin B, a vacuolar cysteine protease (Iqbal, M., et al. J. Med Chem. 38: 2276-2277, 1995; Iqbal, M., et al. Bioorg. Med. Chem. Lett. 6: 287-290, 1996; and Harding, C. V., et al. J. Immuno. 155: 1767-1775, 1995). Furthermore, the order of potency for inhibition of the chymotrypsin-like activity was reported to be Compound A>Compound B>Compound C by using both isolated MCP and intact murine B cells (Iqbal, M., et al. J. Med Chem. 38: 2276-2277, 1995; Iqbal, M., et al. Bioorg. Med. Chem. Lett. 6: 287-290, 1996; and Harding, C. V., et al. J. Immuno. 155: 1767-1775, 1995).

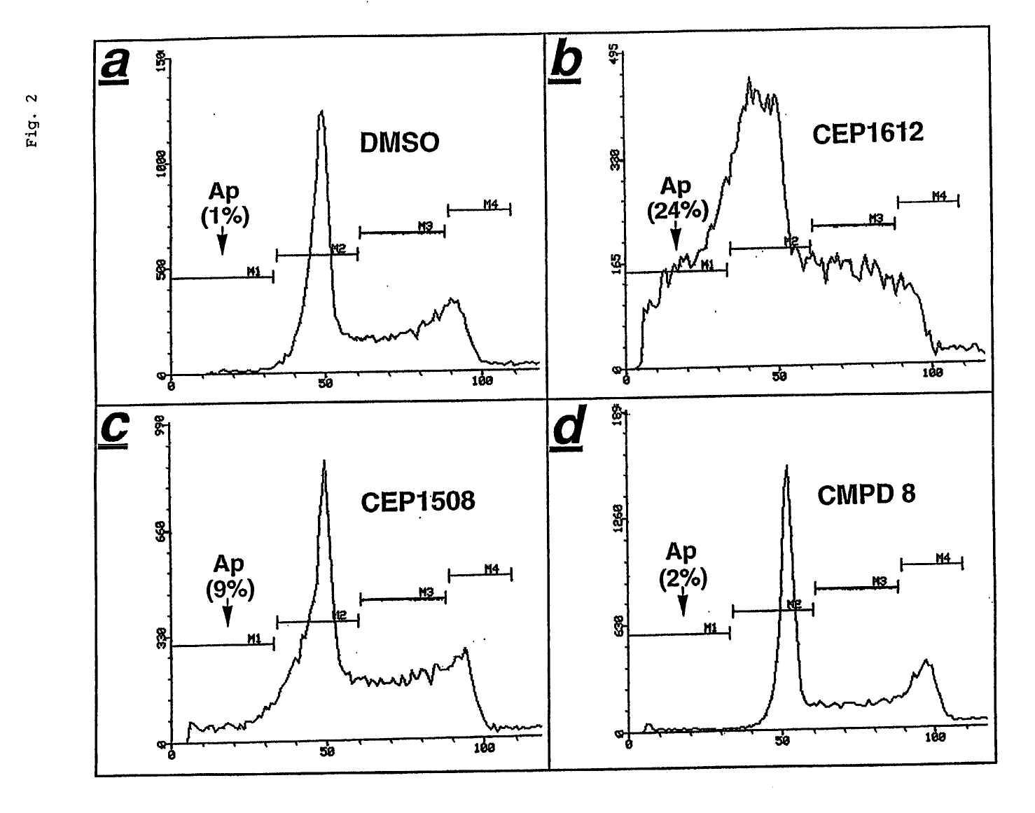

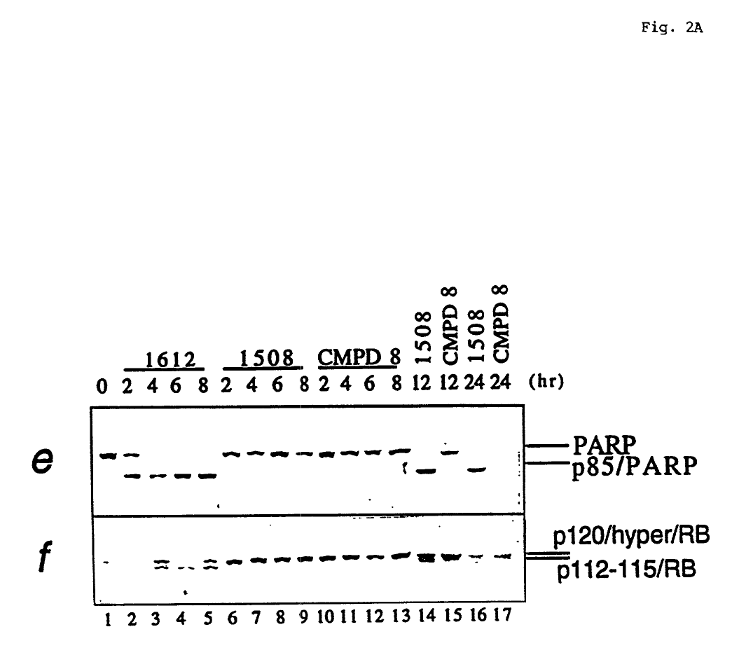

To demonstrate that apoptosis induced by the disclosed MCP inhibitors is due to inhibition of proteasomal enzymatic activity which confers a survival advantage and is not merely due to toxicity caused by the MCP inhibitors, the abilities of Compound A, Compound B, and Compound C to induce apoptosis was investigated. When human Jurkat T cells were treated with 15 μM Compound A for 8 h, there was a 23% increase in the apoptotic population with sub-G₁ DNA content (FIG. 2, panels b vs. a). By comparison, when Compound B was used, only an 8%-increase in sub-G₁ population was detected (FIG. 2, panels c vs. a). Treatment with Compound C (CMPD 8) under the same conditions did not induce apoptosis under these conditions (FIG. 2, panel d).

The ability of these three MCP inhibitors to induce changes in PARP and RB proteins in a parallel experiment was also investigated. When Jurkat T cells were treated with 15 μM Compound A, production of p85/PARP and p 112-115/RB were observed after 2 h (FIG. 2A, e and f, lanes 2-5 vs. 1). However, when these cells were exposed to Compound B at the same concentration, much less p85/PARP and p 112-115/RB were found before 8 h (FIG. 2A,, lanes 6-9 vs. lanes 2-5) but significantly increased after 12 h or longer of treatment (FIG. 2A, lanes 14, 16). Less potent than either of Compounds A or B, Compound C (CMPD 8) only induced little p85/PARP and p112-115/RB after 24 h (FIG. 2A, lane 17). Therefore, based upon these data, the order of apoptosis-inducing potency for these MCP Inhibitors was Compound A>Compound B>Compound C, as judged by induction of sub-G₁ population and changes in PARP and RB proteins (FIGS. 2 and 2A). This rank corresponded exactly to that of the three compounds for inhibition of the chymotrypsin-like activity in isolated proteasomes (Iqbal, M., et al. J. Med Chem. 38: 2276-2277, 1995; Iqbal, M., et al. Bioorg. Med. Chem. Lett. 6: 287-290, 1996) and in intact murine B cells (Harding, C. V., et al. J Immuno. 155: 1767-1775, 1995).

The results presented here support the position that induction of apoptosis by the disclosed MCP inhibitors is due to inhibition of the chymotrypsin-like activity of MCP.

### 4. Example 3: Compound A has Apoptosis-inducing Potency and is Able to Overcome Bcl-2-mediated Protection from Apoptosis

The apoptosis-inducing potency of Compound A was compared with two standard chemotherapeutic anti-cancer agents, etoposide and cisplatin. After treatment with 30 μM Compound A for 3.5 h, nearly 100% of Jurkat cells (data not shown) or Jurkat cells transfected with a control vector (for the below Bcl-2 studies; FIG. 3, panels b vs. a) exhibited apoptotic nuclear changes. By comparison, treatment with 50 μM etoposide for 8 h induced only ˜47% of these cells to undergo apoptosis (FIG. 3, panels c vs. a). Treatment of 10 μM Compound A, but not etoposide or cisplatin, induced apoptosis in human prostate, breast, tongue and brain cancer cells and also in SV40 DNA virus-transformed human fibroblasts (see FIGS. 5-7).

It has been shown that overexpression of Bcl-2 oncoprotein inhibits apoptosis in many cell systems (Miura, M., et al., J. Cell 75: 653-660, 1993). Bcl-2 expression in human Jurkat T cells for inhibition of Compound A-induced apoptosis was investigated. After exposure to 30 μM Compound A for 3.5 h, ˜100% of the Bcl-2-overexpressing Jurkat cells (FIG. 3, panels e vs. d), similar to the vector-transfected cells (FIG. 3, panels b vs. a), exhibited the apoptosis-specific nuclear morphology. In contrast, expression of Bcl-2 protein blocked the apoptotic nuclear changes induced by etoposide (FIG. 3, panels f vs. c) as previously determined by the inventors (An B., et al., Int. J. Mol. Med., In Press) and by others (Miyashita, T., & Reed, J. C. Blood, 81: 151-157, 1993).

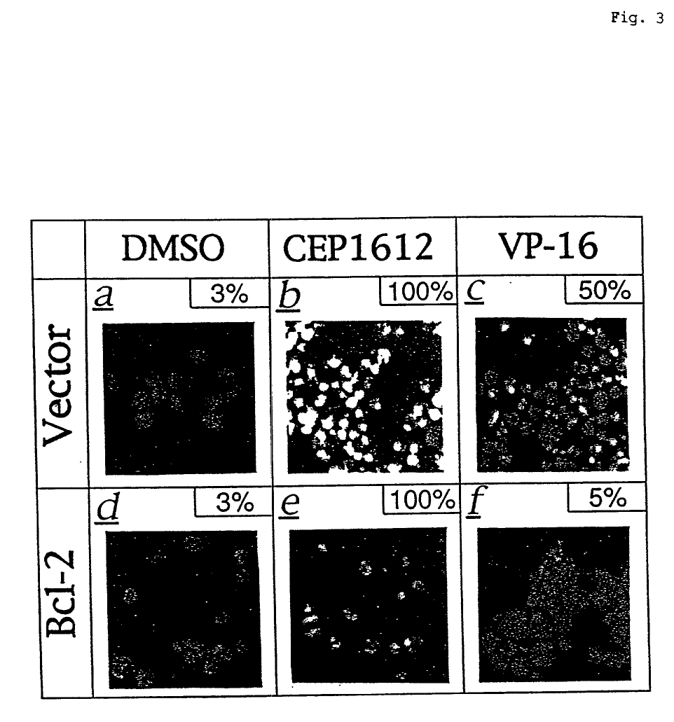

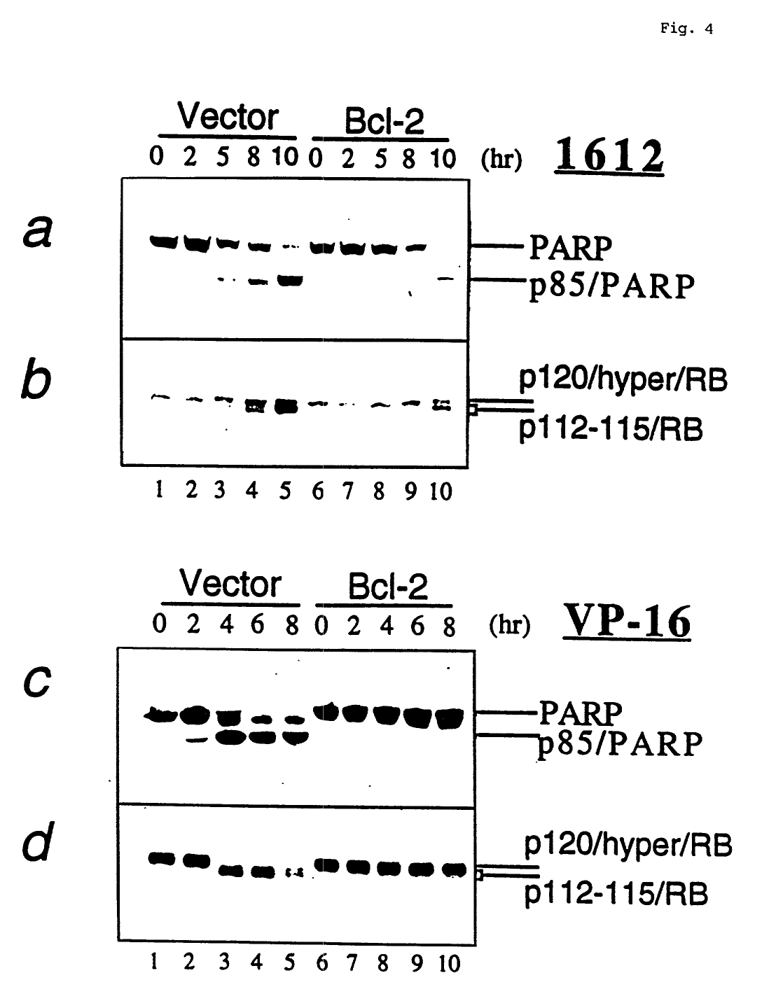

To further confirm that Compound A overcame protection by Bcl-2 from apoptosis, the control vector cells were exposed to 15 μM Compound A for 8 h, a treatment which was less effective than 50 μM etoposide for 8 h, as determined by the percentage of cells exhibiting apoptotic nuclear morphology (30% vs. 47%, respectively). Expression of Bcl-2 did not inhibit the apoptotic nuclear changes induced by the lower concentration of Compound A (data not shown). Consistent with this, overexpression of Bcl-2 also failed to block cleavage of PARP and production of p112-115/RB induced by 15 μM Compound A (FIG. 4, a and b, lanes 8-10 vs. 3-5). In contrast, expression of Bcl-2 inhibited these PARP and RB changes induced by 50 μM etoposide (FIG. 4, c and d, lanes 6-10 vs. lanes 1-5). Based upon these data, the disclosed MCP inhibitors initiate the apoptotic death program through a Bcl-2-independent pathway.

### 5. Example 4: Compound A Induces Apoptosis in Multiple Human Tumor Cell Lines

Most human solid tumors are resistant to treatment with currently-used chemotherapeutic agents. It has been suggested that overexpression of the Bcl-2 oncoprotein contributes to the development of multidrug resistance, at least in human prostate and breast cancers (Harrison et. al., J. Pathol. 175: 7-12, 1995; Desoize et. al., Anticancer Res. 14: 2291-2294, 1994; Kellen et. al., Anticancer Res. 14: 433-436, 1994). Because Compound A was able to overcome Bcl-2-mediated inhibition of apoptosis in human Jurkat T cells (FIGS. 3, 4), Compound A was investigated for its ability to induce apoptosis in human prostate (PC-3, DU145) and breast (MDA-MB-231, MCF-7) cancer cell lines. In these experiments, the efficacy of Compound A was compared with the efficacy of etoposide.

Human prostate cancer PC-3 cells were treated with 10 μM Compound A, etoposide, or an equal percentage of vehicle (DMSO), followed by separation of the attached and detached cell populations. Both attached and detached cell populations were then used for detection of apoptotic nuclear changes. After 36 h treatment with Compound A, ˜50% of PC-3 cells became detached. All the detached PC-3 cells exhibited typical apoptotic nuclear condensation and fragmentation (FIG. 5, panel a). While not wishing to be bound by any particular theory, such cellular detachment is probably triggered by induction of apoptosis, because the remaining attached cells also showed apoptotic nuclear morphology (FIG. 5, panel b). Little or no detachment was observed in PC-3 cells treated with either etoposide or DMSO; consistent with that, all the remaining attached cells contained normal, round nuclei (FIG. 5, panels c and d, respectively).

Similar to PC-3, about half of human prostate cancer DU145 cells became detached after exposure to Compound A for 16 h. Almost all the detached and even the attached DU145 cells exhibited apoptosis-specific nuclear morphology (FIG. 5, panels e, f vs. h). The etoposide treatment of these cells only induced very little detachment, and the remaining attached cells still contained normal nuclei (FIG. 5, panel g).

Detachment was also found in at least half of human breast cancer MDA-MB-231 and MCF-7 cells, after exposure to Compound A for 24 h. Nuclear staining assays demonstrated apoptotic morphology in all of the detached and most of the attached cells (FIG. 5, panels l, j, m and n). Treatment of these two cell lines with etoposide under the same conditions did not induce apoptosis (FIG. 5, panels k, o). Because MDA-MB-231 cells contain a mutant p53 gene (Casey, G., et al. Oncogene, 6: 1791-1797, 1991) and MCF-7 cells express the wild-type p53 (Bartek, J., et al. Oncogene, 5: 893-899, 1990), these data support the position that induction of apoptosis by Compound A is p53-independent.

Treatment with Compound A, but not etoposide or cisplatin, induced cellular detachment and apoptosis in several other human tumor lines, including oral aquamous carcinoma cell line SCC-25 (FIG. 5, panels q-t), glioblastoma cell line SNB-19 (panels u-x) and osteosarcoma cell line U2OS (data not shown). Taken together, these data support the position that Compound A has a greater apoptosis-inducing potency than etoposide and cisplatin, and demonstrate that this MCP inhibitor is able to overcome drug resistance in a variety of human cancer cell lines.

### 6. Example 5: Compound A Induces Apoptosis Selectively in SV40-transformed, But Not in the Parental Normal, Human Fibroblasts

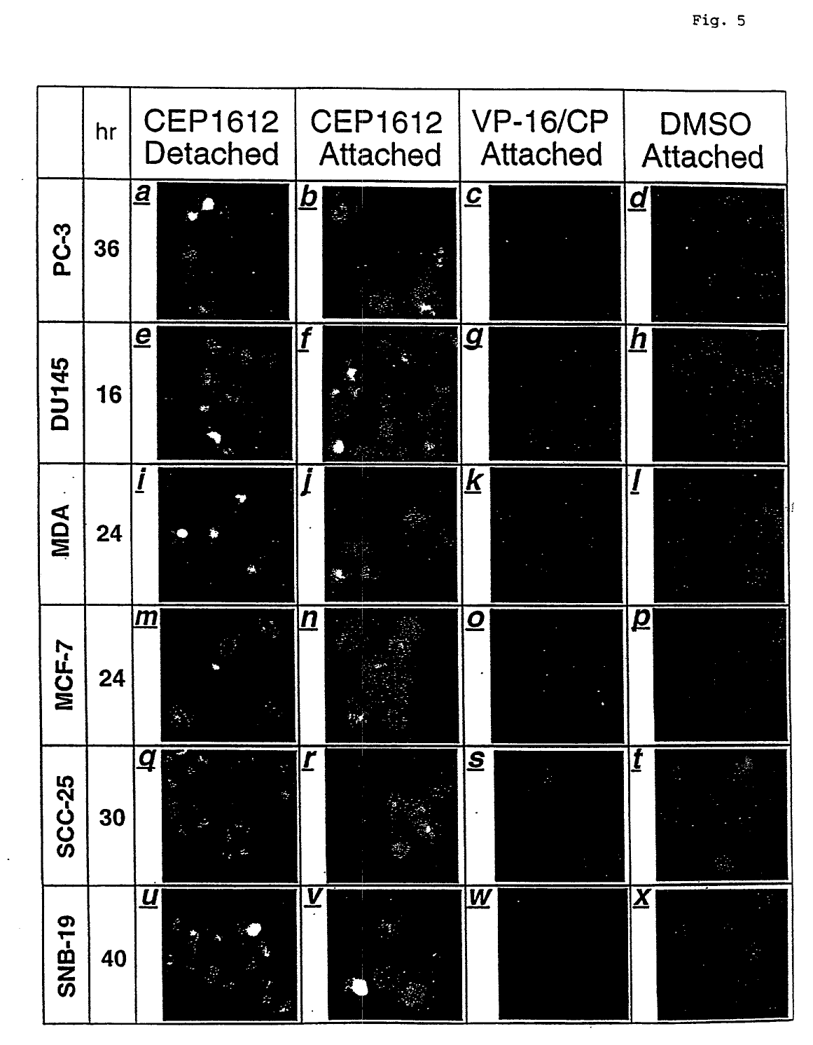

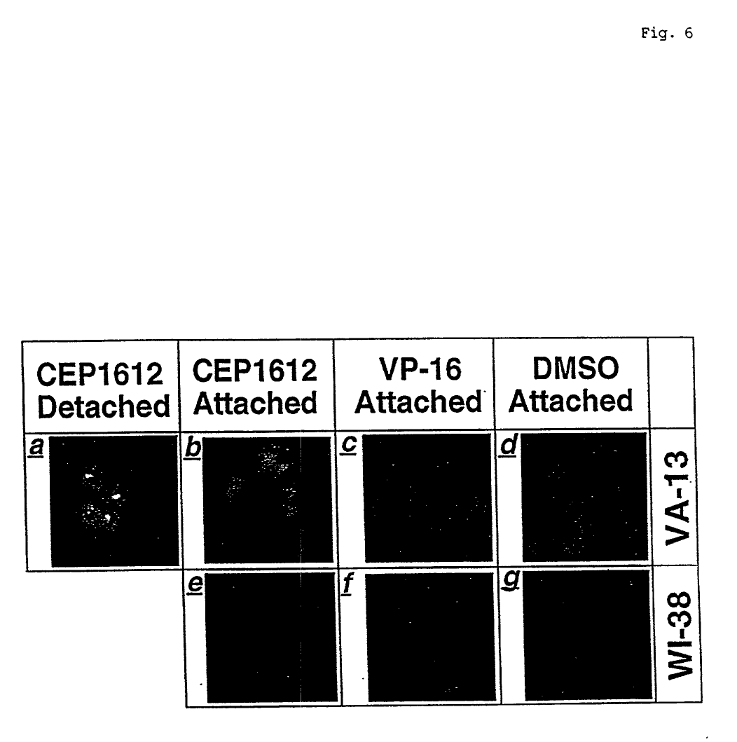

Whether Compound A has, any selectivity in induction of apoptosis between transformed and normal cells was investigated. Normal human fibroblast cell line (WI-38) and its SV40-transformed derivative (WI-38 VA13) were utilized. This pair of cell lines was treated with either 10 μM Compound A, etoposide or DMSO for up to 22 h, followed by separation of the attached and detached cell populations. Compound A treatment induced a detachment in the majority of SV40-transformed cells; all of the detached, and most of the attached, transformed cells exhibited apoptotic nuclear changes (FIG. 6, panels a, b vs. d). In contrast, exposure of normal WI-38 cells to Compound A did not induce either detachment or apoptotic nuclear morphology, although this treatment increased the volume of normal WI-38 cells (panels e vs. g), which, while not wishing to be bound by any particular theory, was probably due to a growth arrest in G₁ (Hinds, P. W., et al. Cell 70: 993-1006, 1992). Treatment with etoposide did not induce apoptosis in either transformed or normal WI-38 cells, although this treatment also increased cellular volumes in both lines (FIG. 6, panels c vs. d and f vs. g).

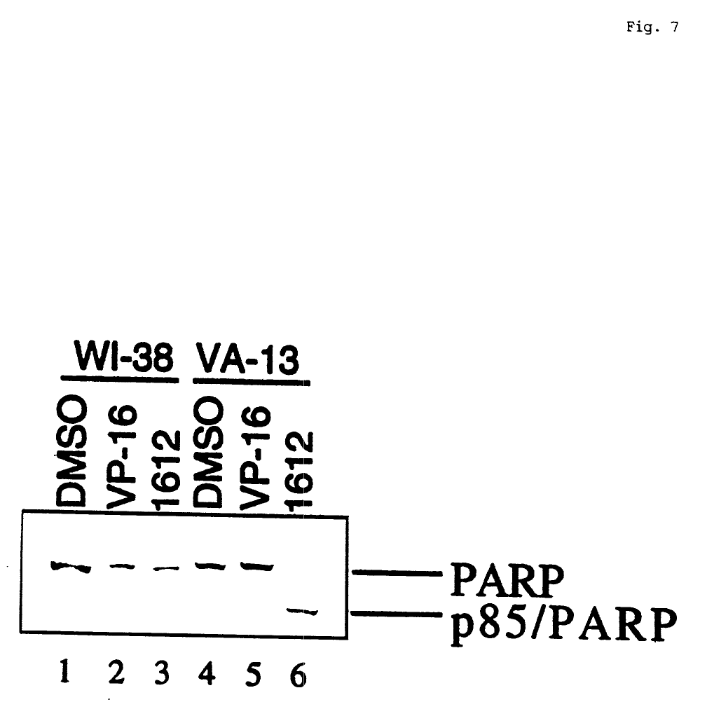

In a parallel comparison experiment, total cell populations (a mixture of both detached and attached cells) were collected after treatment with Compound A, etoposide or DMSO. Whole cell extracts were then prepared from these cells and used for measurement of PARP cleavage. Consistent with the results from nuclear staining assay (FIG. 6), Compound A-induced PARP cleavage was observed only in the transformed (FIG. 7, lanes 6 vs. 4), but not in the normal (FIG. 7, lanes 3 vs. 1; note: 4-fold more protein from WI-38 cells was used in this experiment). Taken together, the data support the position that Compound A selectively induces the apoptotic death process in the SV40-transformed human fibroblasts.

### 7. Example 6: Treatment of Cells with Compound A Induces Accumulation of the Cyclin-dependent Kinase Inhibitors p21 and p27.

The ubiquitin-proteasome pathway has been reported to play an essential role in control of the levels of several cell cycle regulatory proteins, including the cyclin-dependent kinase inhibitors p21 (Blagosklonny, M. V., et al., Biochem. Biophys Res. Comm. 227: 564-569 (1996) and p27 (Pagano, M., et al., Science 269: 682-685 1995). The effect of Compound A on levels of p21 and p27 was examined in human breast cancer MDA-MB-231 cells by western blot. After 6 hours of exposure to 15 μM Compound A, the level of p 21 was increased 45 fold. Levels of p27 were slightly increased after 6 hours and were elevated 3- to 4-fold after 12 or 24 hours. Additionally, a band of 70 kDa whih may represent ubiquinated p70 was also observed. In contrast, etoposide caused only limited accumulation of p21 (≦4-fold) and p27 (≦2-fold) at 17 hours when used at concentrations up to 100 μM.

The accumulation of p21 and p27 following treatment with Compound A was also evaluated in SV-40 transformed and normal WI-38 fibroblasts. Incubation of either normal or transformed WI-38 cells with 10 μM Compound A for 22 hours resulted in a 9-to 10-fold enhancement in p21 levels. The p27 levels in normal cells increased only slightly under these conditions, but p27 levels in SV-40 transformed cells were elevated 8-fold.

### 8. Analysis of In Vitro Results

The foregoing data support the position that Compound A rapidly induced apoptosis in p53-mutant human Jurkat T and HL-60 cells, as evidenced by appearance of the apoptotic population with sub-G₁ DNA content, nuclear condensation and fragmentation, the internucleosomal DNA fragmentation, processing and activation of caspase-3, cleavage of PARP, and dephosphorylation and cleavage of RB (FIG. 1). Addition of YVAD-CMK blocked all of the above apoptotic events (FIG. 1A), confirming the requirement of caspase activation in MCP inhibitor-induced, p53-independent apoptosis. These data are consistent with, and have further extended, the most recent reports from others in which apoptotic death was induced by other MCP inhibitors, including tripeptide aldehydes (LLL, LLnV) or lactacystin (Imajoh, Ohmi, et al. Biochem. Biophy. Res. Commu. 217: 1070-1077, 1995; Shinohara, K., et al. Biochem. J. 317: 385-388, 1996; Drexler, H. C. A. PNAS USA. 94: 855-860, 1997; and Lopes, U. G., et al. J. Biol. Chem. 272: 12893-1896, 1997). The apoptosis-inducing abilities of Compounds A, B and C (FIG. 2) are proportional to their inhibitory potency toward the chymotrypsin-like activity of MCP (Iqbal, M., et al. J. Med Chem. 38: 2276-2277, 1995; Iqbal, M., et al. Bioorg. Med. Chem. Lett. 6: 287-290, 1996). Compound A has a greater apoptosis-inducing potency than two standard chemotherapeutic drugs, etoposide and cisplatin. Consistent with this was the finding that Compound A, but not etoposide or cisplatin, was able to induce apoptosis in Jurkat cells overexpressing Bcl-2 or in multiple human cancer cell lines of prostate, breast, tongue and brain (FIGS. 3-5). Compound A induced apoptosis selectively in the SV40-transformed, but not in the parental normal, human fibroblasts (FIG. 6, 7).

Additionally, Compound A increased levels of the cyclin-dependent kinase inhibitors p21 and p27 in a human breast cell tumor line. Levels of p27 were selectively enhanced in SV40-transformed fibroblasts, but not in the untransformed parental line.

a. Inhibition of the proteasome chymotrypsin-like activity is associated with induction of apoptosis

The foregoing data support the requirement of the proteasome chymotryptic component, and not the trypsin-like component, for cell survival, although a role for the branched chain amino-acid preferring activity cannot be ruled out. The data indicate that the rank in ability to induce apoptosis by Compounds A, B and C in Jurkat T cells (FIG. 2) corresponded exactly to their rank in potency toward inhibition of the proteasome chymotryptic activity (Iqbal, M. et al. J. Med. Chem. 38: 2276-2277, 1995; Iqbal, M. et al. Bioorg. Med. Chem. Lett. 6: 287-290, 1996; and Harding, C. V. et al., J. Immuno. 155: 1767-1775, 1995). In addition, the data support the position that Compound A (Ki=<2nM; Iqbal, M., et al. supra) is a more potent inhibitor than the tripeptide aldehydes (Ki=20 nM; Rock, K. L., et al. Cell. 78:761-771, 1994) toward the proteasome chymotrypsin-like activity. Consistent with this, Compound A has a greater apoptosis-inducing ability than tripeptide aldehydes. For example, treatment with 30 μM Compound A for 3-4 h was sufficient to induce a complete cleavage of PARP in human Jurkat cells of HL-60 cells (FIG. 1A). In contrast, it has been reported that treatment of HL-60 cells with 50 μM LLnV for 6 h induced ˜50% PARP cleavage (Drexler, H. C. A. PNAS USA. 94: 855-860, 1997). These results support the position that the proteasome chymotrypsin-like activity is involved in the cellular survival pathways and that inhibition of this activity leads to induction of apoptosis.

b. Compound A overcomes drug resistance of human cancer cells

Based upon the data presented herein, Compound A is a potent apoptosis inducer and appears to be able to overcome drug resistance of human cancer cells. Compound A, but not etoposide, was able to induce apoptosis in Jurkat T cells overexpressing Bcl-2 (FIG. 3). This was also true even when a lower concentration of Compound A was used as compared to a higher concentration of etoposide (FIG. 4). These data support the position that Compound A induces apoptosis through a novel, Bcl-2-independent pathway. Most of the human cancer cells are resistant to treatment with standard anticancer drugs, such as etoposide or cisplatin (Harrison, D. J. J. Patho. 175: 7-12, 1995; FIG. 5). However, a low concentration of Compound A rapidly activated the apoptotic pathway in all the tested human cancer cell lines of prostate, breast, tongue and brain (FIG. 5). In addition to the independence of Bcl-2, MCP inhibitor-induced apoptosis is also p53-independent, which is different from proteasome-mediated p53-dependent apoptosis reported most recently (Lopes, U. G., J. Biol. Chem. 272: 12893-1896, 1997). These properties support the position that the disclosed MCP inhibitors are novel anticancer agents for the treatment of human cancers, especially those overexpressing Bcl-2 and/or lacking p53.

c. Compound A selectively induces apoptosis in SV40-transformed but not normal human fibroblasts

The potency of Compound A was compared between normal WI-38 and its SV40-transformed derivative (WI-38 VA-13) cell lines. Compound A treatment was found to induce detachment and apoptosis preferably in SV40-transformed cells (FIG. 6). Consistent with this, Compound A treatment induced cleavage of PARP only in the transformed, but not in the normal, WI-38 cells (FIG. 7). These data support the position that Compound A-mediated killing is not a cytotoxic effect, and further suggest that Compound A may have a tumor-selective killing ability. The differential activity of Compound A in normal and transformed cells can not be explained by differences in proliferation rates, as these were similar for both WI-38 and WI-36 VA-13 cells.

d. Treatment of cells with Compound A induces accumulation of the cyclin-dependent kinase inhibitors p21 and p27

The ability of compound A to modulate p21 and p27 levels is consistent with the reported role of the proteasome in control of the cyclin-dependent kinase inhibitors (Blagosklonny, M. V., et al., supra; Pagano, M. et al., supra). The selective accumulation of p27 in SV40-transformed WI-38 fibroblasts, but not in the parental cell line, following treatment Compound A selectively induced apoptosis in the transformed cells. This observation is consistent with the hypothesis that accumulation of p27 above a critical threshold concentration results in apoptosis.

### 9. Example 7: In vivo Investigation

a. Materials: MCP inhibitors used for in vivo studies (Compounds D and E) were each formulated in 25% Solutol.

b. Cell line: The murine melanoma cell line, B16-F0, was grown at 37° C. in a humidified incubator, with a 95% air/5% CO₂ atmosphere, in Dulbecco's modified Eagle's medium with 4.5 g/l glucose (Cellgro/Mediatech, Washington, D.C.) containing 10% fetal bovine serum (Hyclone Labs, Logan, Utah), 2 mM glutamine (GibcoBRL, Long Island, N.Y.), penicillin (100 I.U./mL) (GibcoBRL), and streptomycin (100 μg/mL) (GibcoBRL). The cells were determined to be free of mycoplasma and rodent viruses (MAP testing). Exponentially growing cells were harvested using 5 mL of warm trypsin/EDTA (0.05%, 0.5 mM)((GibcoBRL). The total volume was brought up to 10 mL with Complete Medium to neutralize trypsin and cells were counted with a hemocytometer. The cells were then collected by brief centrifugation and the cell pellet was resuspended in Phosphate Buffered Saline (GibcoBRL) to achieve the final concentration of 1×10⁷ live cells/ml.

c. Animals: Female C57BL mice (20-25 g) obtained from Harlan Sprague Dawley, Indianapolis, Ind. were maintained five mice/cage and given a commercial diet and water ad libitum. Animals were housed under humidity- and temperature-controlled conditions and light/dark cycle was set at 12-hour intervals. Mice were quarantined for one week before experimental manipulation.

d. Tumor cell implantation and growth: Exponentially growing B16-F0 cells, cultured as described above, were harvested and injected (1×10⁶ cells/mouse) into the right flank of the mice. Fifty (50) animals bearing tumors of 0.01-0.3 cm³ size were divided into 5 groups of 10 animals each. Compounds were administered at 10 mg/kg/day, ip; Vehicle (25% Solutol) was administered at 1 ml/kg/day, ip.

e. Tumor measurements: Tumors were measured using a vernier caliper every 2 to 3 days. Tumor volume was calculated using the formula:

V(cm)³=0.5236×length(cm)×width(cm)[(length(cm)+width(cm))/2].

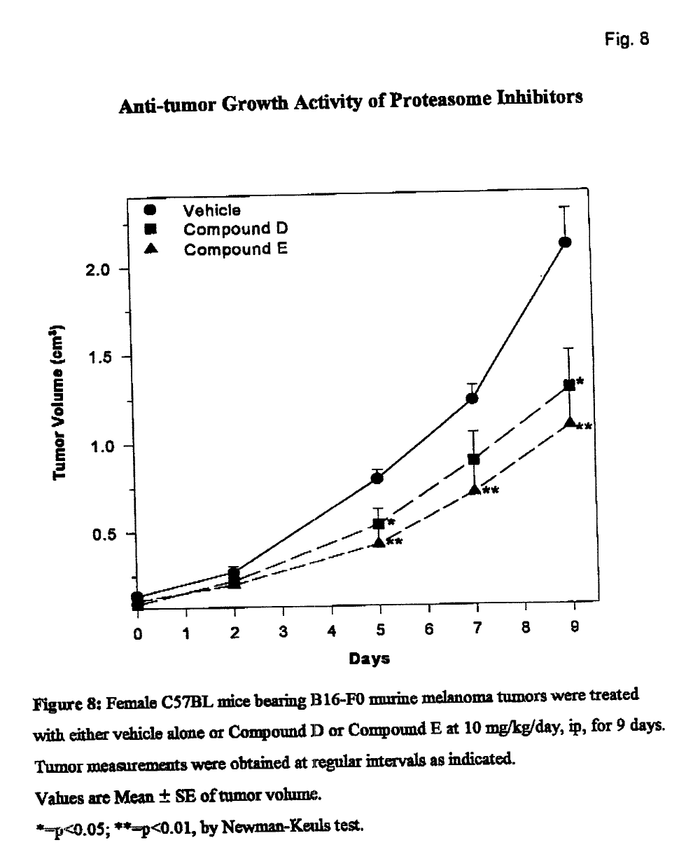

Results from the in vivo studies are presented in FIG. 8.

The results show that Compounds D and E inhibit melanoma tumor growth.

### 10. Example 8: In vivo Anti-tumor Efficacy of Compound I and Compound J on the Growth of Lewis Lung Carcinoma Xenografts in Athymic Nude Mice

Female athymic nude mice were injected s.c. with 1×10⁶ Lewis Lung carcinoma cells into the right rear flank. Upon tumors achieving 150 to 200 mm³ in volume, mice were randomized into groups of ten animals each and dosing commenced with Compound I (2 mg/kg, s.c., QD, 5 days a week), Compound J (3 mg/kg, s.c., QD, 5 days a week), or vehicle alone (30% Solutol) for a total of 12 days. Tumor measurements (volume) were determined with vernier calipers in two dimensions every two to three days. Statistical analyses of drug-associated anti-tumor efficacy relative to vehicle-treated controls were conducted using the Mann-Whitney Rank sum test.

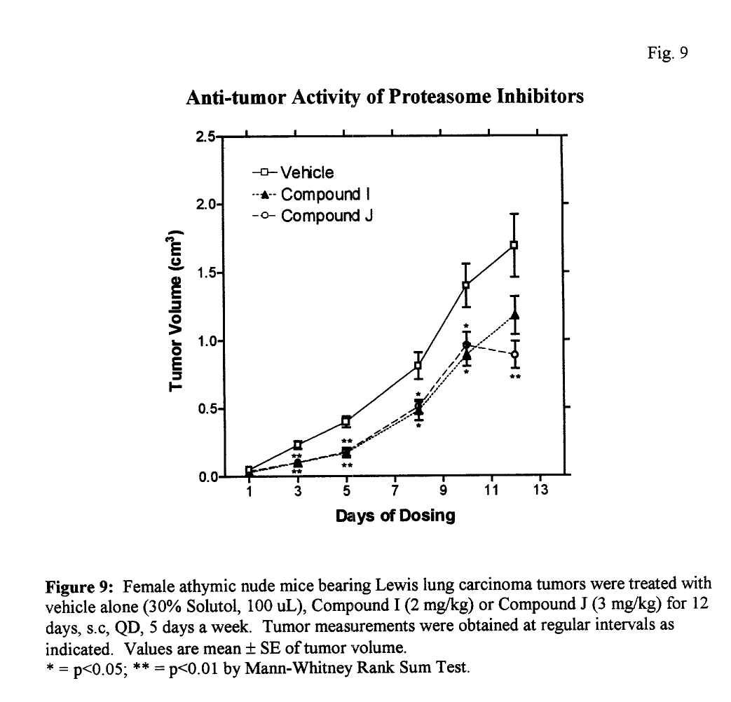

Results are presented in FIG. 9.

The results show that Compounds I and J inhibit lung carcinoma tumor growth.

### 11. Example 9: In vivo Anti-tumor Efficacy of Compound I on the Growth of AT-2 Rat Prostatic Carcinoma Xenografts in Athymic Nude Mice

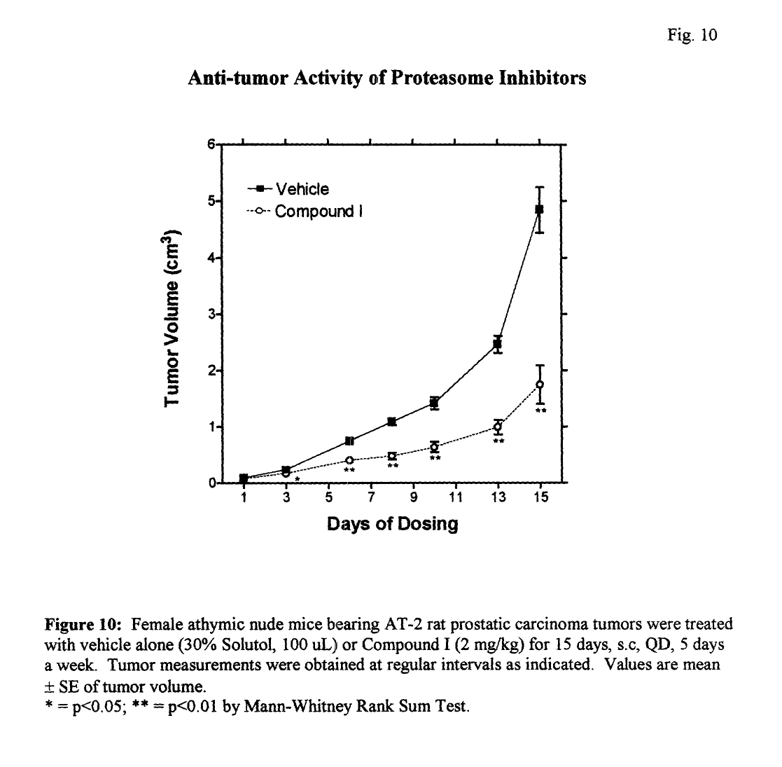

Female athymic nude mice were injected s.c. with 1×10⁶ AT-2 rat prostatic carcinoma cells into the right rear flank. Mice were randomized into groups of ten animals each and dosing commenced with Compound I (2 mg/kg, s.c., QD, 5 days a week) or vehicle alone (30% Solutol) for a total of 15 days. Tumor measurements (volume) were determined with vernier calipers in two dimensions every two to three days. Statistical analyses of drug-associated anti-tumor efficacy relative to vehicle-treated controls were conducted using the Mann-Whitney Rank sum test. Results are presented in FIG. 10.

### 12. Example 10: Effect of Compound I and Compound J on the In vitro Viability of Human and Rodent Tumor Cell Lines

Cells were initially seeded in 96-well plates at varying density, then assayed using the Calcein-AM viability assay after 24 hours to determine the optimal final density for each cell type. Cells were then seeded in 96-well plates at this density in 100 μL of the proper cell media as follows:

| Cell Line | Optimal Density | Culture Media |
|---|---|---|
| DU145 prostatic carcinoma | 2500 cells/well | DMEM/5% FBS |
| PANC-1 pancreatic carcinoma | 4000 cells/well | MEM/5% FBS |
| SKMEL-5 melanoma | 3500 cells/well | DMEM/5% EBS |
| OVCAR-3 ovarian carcinoma | 5000 cells/well | RPMI 1640/5% FBS |
| MCF-7 breast carcinoma | 5000 cells/well | DMEM/5% FBS |
| AT-2 (rat) prostatic carcinoma | 2500 cells/well | RPMI 1640/5% FBS |
| Lewis lung (murine) lung | 3000 cells/well | DMEM/5% FBS |
| carcinoma | | |

Serial dilutions of the compounds were made so that concentrations were twice the desired concentration to be evaluated. When 100 μL of the dilution was then added to the cells plated in 100 μL of media, a final concentration of 0, 11.7, 46.9, 187.5, 375 and 750 nM was obtained. Compounds were added to the plates three to four hours after seeding the cells, then the plates were incubated at 37° C. for the desired time point (generally one, two, or three day incubations).

Calcein-AM viability assays were conducted at the desired time points as follows. Media were aspirated using a manifold and metal plate to leave approximately 50 μL/well. The wells were then washed three times with 200 μL DPBS (Gibco), aspirating each time with the manifold to leave 50 μL/well. A 8 μM solution of Calcein-AM (Molecular Probes) in DPBS was prepared and 150 μL was added to each well. The plates were then incubated at 37° C. for 30 minutes. After incubation, calcein was aspirated with the manifold and cells were washed with 200 μL DPBS as before. After the final aspiration, fluorescence was measured using a Cytofluor 2300 fluorescence plate reader. Negative controls contained media but no cells. All studies were conducted in triplicate in two independent experiments.

Results are presented in Table 3 below:

TABLE 3

| | | Compound I | Compound J |
|---|---|---|---|
| Cell Type | Time Point (h) | IC₅₀ (nM) | IC₅₀ (nM) |
| PANC-1 | 24 | >1000 | >1000 |
| | 72 | 282 | 185 |
| SKMEL-5 | 24 | 520 | 427 |
| | 72 | 85 | 82 |
| OVCAR-3 | 24 | >1000 | >1000 |
| | 72 | 69 | 29 |
| MCF-7 | 24 | >1000 | >1000 |
| | 72 | 642 | 289 |
| Lewis Lung | 24 | 435 | 411 |
| | 72 | 578 | 505 |
| AT-2 | 24 | >1000 | >1000 |
| | 72 | >1000 | >1000 |
| DU-145 | 24 | 942 | 385 |
| | 72 | 293 | 167 |

It is intended that each of the patents, applications, and printed publications mentioned in this patent document be hereby incorporated by reference in their entirety.

Those skilled in the art will appreciate that numerous changes and modifications may be made to the preferred embodiments of the invention and that such changes and modifications may be made without departing from the spirit of the invention. It is therefore intended that any appended claims cover all equivalent variations as fall within the true spirit and scope of the invention.

What is claimed is:

1. A method for causing the death of transformed cells comprising contacting said cells with a compound of formula: 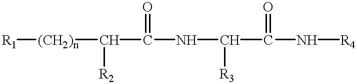
wherein:
R₁ is selected from the group consisting of —C≡N, —C(═O)OR₉, phthalimido, —NH— SO₂R₉, and —NH—J;
R₂ is selected from the group consisting of H, hydroxyl, alkyl having from one to ten carbons, and cycloalkyl having from three to seven carbons;
R₃ is selected from the group consisting of —(CH₂)ₘ—NH—C(═N—R₅)—NH₂, —R₆—NO₂, —R₆—J, and —R₆—C≡N;
R₄ is —CH(CH₂—R₇)—Q;
Q is selected from the group consisting of —CH—R₈, —C(═O)CH₃, —C(═O)CH₂Cl, —C(═O)CH₂Br, —C(═O)CH₂F, —C(═O)CHF₂, —C(═O)CF₃, —C(═O)C(═O)R₇, —C(═O)C(═O)NH—R₇, —C(═O)CO₂—R₇, —C(═O)CO₂H, —B(OH)₂, 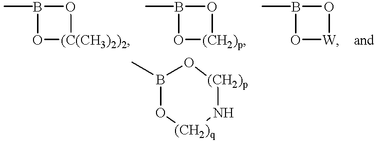
where p and q, independently, are 2 or 3;
W is cycloalkyl;
R₅ is selected from the group consisting of —NO₂, —C≡N, and —J;
R₆ is —(CH₂)ₘ—NH—C(═NH)—NH—;
R₇ is selected from the group consisting of phenyl, and alkyl having from one to eight carbons, said alkyl group being optionally substituted with one or more halogen atoms, aryl, or heteroaryl groups;
R₈ is selected from the group consisting of ═O, ═N—NHC(═O)—NH₂, ═N—OH, ═N—OCH₃, ═N—O—CH₂—C₆H₅, ═NNH—C(═S)—NH₂ and ═N—NH—J;
R₉ is selected from the group consisting of hydrogen and alkyl having from one to six carbons, said alkyl group being optionally substituted with one or more halogen atoms, aryl or heteroaryl groups;
J is a protecting group;
n is an integer from 3 to 10; and
m is an integer from 2 to 5.

2. The method of claim 1 wherein R₁ is —C≡N, —C(═O)OCH₃, phthalimido or —NH—SO₂CF₃.

3. The method of claim 1 wherein R₂ is H or cyclopentyl.

4. The method of claim 1 wherein R₃ is —(CH₂)₃—NH—C(═N—R₅)—NH₂.

5. The method of claim 1 wherein Q is —CH—R₈, —B(OH)₂, —C(═O)C(═O)NH—R₇, or has the structure: 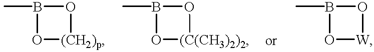
where W is pinane.

6. The method of claim 1 wherein R₅ is —NO₂, —C≡N, —PMC, —MTR, —MTS, or Tos.

7. The method of claim 1 wherein R₇ is —CH(CH₃)₂, —(CH₂)₂—CH₃, —CH₂—CH₃, or —C₆H₅.

8. The method of claim 1 wherein R₈ is ═O, ═N—OH, ═N—O—CH₂—C₆H₅, ═NNH—C(═O)—NH₂or ═NNH—C(═S)—NH₂.

9. The method of claim 1 wherein R₁ is —C(═O)OCH₃, phthalimido or —NH—SO₂CF₃; R₂ is cyclopentyl; R₃ is —(CH₂)₃—NH—C(═N—NO₂)—NH₂; R₇ is —CH(CH₃)₂; and R₈ is ═O.

10. The method of claim 1 wherein R₁ is —C≡N; R₂ is cyclopentyl; R₃ is —(CH₂)₃—NH—C(═N—NO₂)—NH₂ or —(CH₂)₃—NH—C(═N—J)—NH₂; R₇ is —CH(CH₃)₂; and R₈ is ═O.

11. The method of claim 1 wherein R₁ is C≡N; R₂ is cyclopentyl; R₃ is —(CH₂)₃—NH—C(═N—NO₂)—NH₂ or —(CH₂)₃—NH—C(═N—J)—NH₂; R₇ is —CH(CH₃)₂; Q is —CH—R₈; and R₈ is ═N—NHC(═O)—NH₂, ═N—OH, ═N—OCH₃, or ═N—O—CH₂—C₆H₅.

12. The method of claim 1 wherein R₁, R₂, R₃ and R₄ are selected from the group of substituents shown for the compounds in Table 1.

13. The method of claim 1 wherein said compound is selected from the group consisting of compounds A-J shown in Table 1.

14. The method of any one of claims 1-13 wherein said compound is administered to a mammal.

15. The method of claim 14 wherein said mammal is a human.

16. The method of claim 15 wherein said transformed cells are breast cancer cells, prostate cancer cells, tongue cancer cells, brain cancer cells, lung cancer cells, pancreatic cancer cells, ovarian cancer cells, or skin cancer cells.

17. The method of claim 1 wherein said transformed cells overproduce Bcl2 protein, and/or lack p53 protein.

18. A method for treating a patient having a disease, said disease being characterized by the presence of transformed cells, comprising administering to said patient a compound of Formula: 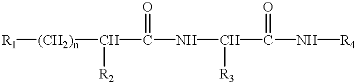
wherein:
R₁ is selected from the group consisting of —C≡N, —C(═O)OR₉, phthalimido, —NH— SO₂R₉, and —NH—J;
R₂ is selected from the group consisting of H, hydroxyl, alkyl having from one to ten carbons, and cycloalkyl having from three to seven carbons;
R₃ is selected from the group consisting of —(CH₂)ₘ—NH—C(═N—R₅)—NH₂, —R₆—NO₂, —R₆—J, and —R₆—C≡N;
R₄ is —CH(CH₂—R₇)—Q;
Q is selected from the group consisting of —CH—R₈, —C(═O)CH₃, —C(═O)CH₂Cl, —C(═O)CH₂Br, —C(═O)CH₂F, —C(═O)CHF₂, —C(═O)CF₃, —C(═O)C(═O)R₇, —C(═O)C(═O)NH—R₇, —C(═O)CO₂—R₇, —C(═O)CO₂H, —B(OH)₂, 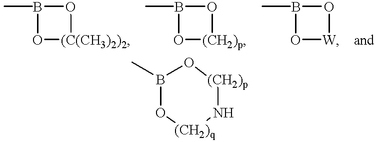
where p and q, independently, are 2 or 3;
W is cycloalkyl;
R₅ is selected from the group consisting of —NO₂, —C═N, and —J;
R₆ is —(CH₂)ₘ—NH—C(═NH)—NH—;
R₇ is selected from the group consisting of phenyl, and alkyl having from one to eight carbons, said alkyl group being optionally substituted with one or more halogen atoms, aryl, or heteroaryl groups;
R₈ is selected from the group consisting of ═O, ═N—NHC(═O)—NH₂, ═N—OH, ═N—OCH₃, ═N—O—CH₂—C₆H₅, ═NNH—C(═S)—NH₂ and ═N—NH—J;
R₉ is selected from the group consisting of hydrogen and alkyl having from one to six carbons, said alkyl group being optionally substituted with one or more halogen atoms, aryl or heteroaryl groups;
J is a protecting group;
n is an integer from 3 to 10; and
m is an integer from 2 to 5.

19. The method of claim 18 wherein R₁ is —C≡N, —C(═O)OCH₃, phthalimido or —NH—SO₂CF₃.

20. The method of claim 18 wherein R₂ is H or cyclopentyl.

21. The method of claim 18 wherein R₃ is —(CH₂)₃—NH—C(═N—R₅)—NH₂.

22. The method of claim 18 wherein Q is —CH—R₈, —B(OH)₂, —C(═O)C(═O)NH—R₇, or has the structure: 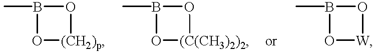
where W is pinane.

23. The method of claim 18 wherein R₅ is —NO₂, —C≡N, —PMC, —MTR, —MTS, or Tos.

24. The method of claim 18 wherein R₇ is —CH(CH₃)₂, —(CH₂)₂—CH₃, —CH₂—CH₃, or —C₆H₅.

25. The method of claim 18 wherein R₈ is ═O, ═N—OH, ═N—O—CH₂—C₆H₅, ═NNH—C(═O)—NH₂ or ═NNH—C(═S)—NH₂.

26. The method of claim 18 wherein R₁ is —C(═O)OCH₃, phthalimido or —NH—SO₂CF₃; R₂ is cyclopentyl; R₃ is —(CH₂)₃—NH—C(═N—NO₂)—NH₂; R₇ is —CH(CH₃)₂; and R₈ is ═O.

27. The method of claim 18 wherein R₁ is —C≡N; R₂ is cyclopentyl; R₃ is —(CH₂)₃—NH—C(═N—NO₂)—NH₂ or —(CH₂)₃—NH—C(═N—J)—NH₂; R₇ is —CH(CH₃)₂; and R₈ is ═O.

28. The method of claim 18 wherein R₁ is C≡N; R₂ is cyclopentyl; R₃ is —(CH₂)₃—NH—C(═N—NO₂)—NH₂ or —(CH₂)₃—NH—C(═N—J)—NH₂; R₇ is —CH(CH₃)₂; Q is —CH—R₈; and R₈ is ═N—NHC(═O)—NH₂, ═N—OH, ═N—OCH₃, or ═N—O—CH₂—C₆H₅.

29. The method of claim 18 wherein R₁, R₂, R₃ and R₄ are selected from the group of substituents shown for the compounds in Table 1.

30. The method of claim 18 wherein said compound is selected from the group consisting of compounds A-J shown in Table 1.

31. The method of any one of claims 18-30 wherein said patient is a human.

32. The method of claim 31 wherein said transformed cells are breast cancer cells, prostate cancer cells, tongue cancer cells, brain cancer cells, lung cancer cells, pancreatic cancer cells, ovarian cancer cells, or skin cancer cells.

33. The method of claim 18 wherein said transformed cells overproduce Bcl2 protein, and/or lack p53 protein.

34. A method for inducing apoptosis in cells comprising contacting said cells with a compound of formula: 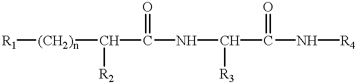
wherein:
R₁ is selected from the group consisting of —C≡N, —C(═O)OR₉, phthalimido, —NH— SO₂R₉, and —NH—J;
R₂ is selected from the group consisting of H, hydroxyl, alkyl having from one to ten carbons, and cycloalkyl having from three to seven carbons;
R₃ is selected from the group consisting of —(CH₂)ₘ—NH—C(═N—R₅)—NH₂, —R₆—NO₂, —R₆—J, and —R₆—C═N;
R₄ is —CH(CH₂—R₇)—Q,
Q is selected from the group consisting of —CH—R₈, —C(═O)CH₃, —C(═O)CH₂Cl, —C(═O)CH₂Br, —C(═O)CH₂F, —C(═O)CHF₂, —C(═O)CF₃, —C(═O)C(═O)R₇, —C(═O)C(═O)NH—R₇, —C(═O)CO₂—R₇, —C(═O)CO₂H, —B(OH)₂, 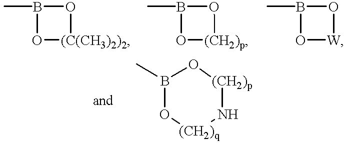
where p and q, independently, are 2 or 3;
W is cycloalkyl;
R₅ is selected from the group consisting of —NO₂, —C═N, and —J;
R₆ is —(CH₂)ₘ—NH—C(═NH)—NH—;
R₇ is selected from the group consisting of phenyl, and alkyl having from one to eight carbons, said alkyl group being optionally substituted with one or more halogen atoms, aryl, or heteroaryl groups;
R₈ is selected from the group consisting of ═O, ═N—NHC(═O)—NH₂, ═N—OH, ═N—OCH₃, ═N—O—CH₂—C₆H₅, ═NNH—C(═S)—NH₂ and ═N—NH—J;
R₉ is selected from the group consisting of hydrogen and alkyl having from one to six carbons, said alkyl group being optionally substituted with one or more halogen atoms, aryl or heteroaryl groups;
J is a protecting group;
n is an integer from 3 to 10; and
m is an integer from 2 to 5.

35. The method of claim 34 wherein R₁ is —C≡N, —C(═O)OCH₃, phthalimido or —NH—SO₂CF₃.

36. The method of claim 34 wherein R₂ is H or cyclopentyl.

37. The method of claim 34 wherein R₃ is —(CH₂)₃—NH—C(═N—R₅)—NH₂.

38. The method of claim 34 wherein Q is —CH—R₈, —B(OH)₂, —C(═O)C(═O)NH—R₇, or has the structure: 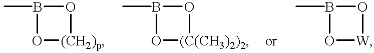
where W is pinane.

39. The method of claim 34 wherein R₅ is —NO₂, —C═N, —PMC, —MTR, —MTS, or Tos.

40. The method of claim 34 wherein R₇ is —CH(CH₃)₂, —(CH₂)₂—CH₃, —CH₂—CH₃, or —C₆H₅.

41. The method of claim 34 wherein R₈ is ═O, ═N—OH, ═N—O—CH₂—C₆H₅, ═NNH—C(═O)—NH₂ or ═NNH—C(═S)—NH₂.

42. The method of claim 34 wherein R₁ is —C(═O)OCH₃, phthalimido or —NH—SO₂CF₃; R₂ is cyclopentyl; R₃ is —(CH₂)₃—NH—C(═N—NO₂)—NH₂; R₇ is —CH(CH₃)₂; and R₈ is ═O.

43. The method of claim 34 wherein R₁ is —C≡N; R₂ is cyclopentyl; R₃ is —(CH₂)₃—NH—C(═N—NO₂)—NH₂ or —(CH₂)₃—NH—C(═N—J)—NH₂; R₇ is —CH(CH₃)₂; and R₈ is ═O.

44. The method of claim 34 wherein R₁ is C≡N; R₂ is cyclopentyl; R₃ is —(CH₂)₃—NH—C(═N—NO₂)—NH₂ or —(CH₂)₃—NH—C(═N—J)—NH₂; R₇ is —CH(CH₃)₂; Q is —CH—R₈; and R₈ is ═N—NHC(═O)—NH₂, ═N—OH, ═N—OCH₃, or ═N—O—CH₂—C₆H₅.

45. The method of claim 34 wherein R₁, R₂, R₃ and R₄ are selected from the group of substituents shown for the compounds in Table 1.

46. The method of claim 34 wherein said compound is selected from the group consisting of compounds A-J shown in Table 1.

47. The method of any one of claims 34-36 wherein said compound is administered to a mammal.

48. The method of claim 47 wherein said mammal is a human.

49. The method of claim 48 wherein said transformed cells are breast cancer cells, prostate cancer cells, tongue cancer cells, brain cancer cells, lung cancer cells, pancreatic cancer cells, ovarian cancer cells, or skin cancer cells.

50. The method of claim 34 wherein said transformed cells overproduce Bcl2 protein, and/or lack p53 protein.

51. A method for inhibiting proliferation of transformed cells comprising contacting said cells with a compound of formula: 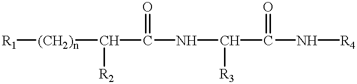
wherein:
R₁ is selected from the group consisting of —C≡N, —C(═O)OR₉, phthalimido, —NH— SO₂R₉, and —NH—J;
R₂ is selected from the group consisting of H, hydroxyl, alkyl having from one to ten carbons, and cycloalkyl having from three to seven carbons;
R₃ is selected from the group consisting of —(CH₂)ₘ—NH—C(═N—R₅)—NH₂, —R₆—NO₂, —R₆—J, and —R₆—C═N;
R₄ is —CH(CH₂—R₇)—Q;
Q is selected from the group consisting of —CH—R₈, —C(═O)CH₃, —C(═O)CH₂Cl, —C(═O)CH₂Br, —C(═O)CH₂F, —C(═O)CHF₂, —C(═O)CF₃,—C(═O)C(═O)R₇, —C(═O)C(═O)NH—R₇, —C(═O)CO₂—R₇, —C(═O)CO₂H, —B(OH)₂, 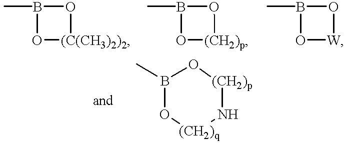
where p and q, independently, are 2 or 3;
W is cycloalkyl;
R₅ is selected from the group consisting of —NO₂, —C═N, and —J;
R₆ is —(CH₂)ₘ—NH—C(═NH)—NH—;
R₇ is selected from the group consisting of phenyl, and alkyl having from one to eight carbons, said alkyl group being optionally substituted with one or more halogen atoms, aryl, or heteroaryl groups;
R₈ is selected from the group consisting of ═O, ═N—NHC(═O)—NH₂, ═N—OH, ═N—OCH₃, ═N—O—CH₂—C₆H₅, ═NNH—C(═S)—NH₂ and ═N—NH—J;
R₉ is selected from the group consisting of hydrogen and alkyl having from one to six carbons, said alkyl group being optionally substituted with one or more halogen atoms, aryl or heteroaryl groups;
J is a protecting group;
n is an integer from 3 to 10; and
m is an integer from 2 to 5.

52. The method of claim 51 wherein R₁ is —C≡N, —C(═O)OCH₃, phthalimido or —NH—SO₂CF₃.

53. The method of claim 51 wherein R₂ is H or cyclopentyl.

54. The method of claim 51 wherein R₃ is —(CH₂)₃—NH—C(═N—R₅)—NH₂.

55. The method of claim 51 wherein Q is —CH—R₈, —B(OH)₂, —C(═O)C(═O)NH—R₇, or has the structure: 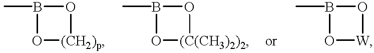
where W is pinane.

56. The method of claim 51 wherein R₅ is —NO₂, —C≡N, —PMC, —MTR, —MTS, or Tos.

57. The method of claim 51 wherein R₇ is —CH(CH₃)₂, —(CH₂)₂—CH₃, —CH₂—CH₃, or —C₆H₅.

58. The method of claim 51 wherein R₈ is ═O, ═N—OH, ═N—O—CH₂—C₆H₅, ═NNH—C(═O)—NH₂ or ═NNH—C(═S)—NH₂.

59. The method of claim 51 wherein R₁ is —C(═O)OCH₃, phthalimido or —NH—SO₂CF₃; R₂ is cyclopentyl; R₃ is —(CH₂)₃—NH—C(═N—NO₂)—NH₂; R₇ is —CH(CH₃)₂; and R₈ is ═O.

60. The method of claim 51 wherein R₁ is —C≡N; R₂ is cyclopentyl; R₃ is —(CH₂)₃—NH—C(═N—NO₂)—NH₂ or —(CH₂)₃—NH—C(═N—J)—NH₂; R₇ is —CH(CH₃)₂; and R₈ is ═O.

61. The method of claim 51 wherein R₁ is C≡N; R₂ is cyclopentyl; R₃ is —(CH₂)₃—NH—C(═N—NO₂)—NH₂ or —(CH₂)₃—NH—C(═N—J)—NH₂; R₇ is —CH(CH₃)₂; Q is —CH—R₈; and R₈ is ═N—NHC(═O)—NH₂, ═N—OH, ═N—OCH₃, or ═N—O—CH₂—C₆H₅.

62. The method of claim 51 wherein R₁, R₂, R₃ and R₄ are selected from the group of substituents shown for the compounds in Table 1.

63. The method of claim 51 wherein said compound is selected from the group consisting of compounds A-J shown in Table 1.

64. The method of any one of claims 51-63 wherein said compound is administered to a mammal.

65. The method of claim 64 wherein said mammal is a human.

66. The method of claim 65 wherein said transformed cells are breast cancer cells, prostate cancer cells, tongue cancer cells, brain cancer cells, lung cancer cells, pancreatic cancer cells, ovarian cancer cells, or skin cancer cells.

67. The method of claim 51 wherein said transformed cells overproduce Bcl2 protein, and/or lack p53 protein.

68. A method for inhibiting the growth of a tumor comprising contacting said tumor with a compound of formula:: 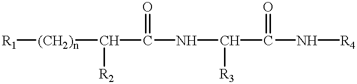
wherein:
R₁ is selected from the group consisting of —C≡N, —C(═O)OR₉, phthalimido, —NH— SO₂R₉, and —NH—J;
R₂ is selected from the group consisting of H, hydroxyl, alkyl having from one to ten carbons, and cycloalkyl having from three to seven carbons;
R₃ is selected from the group consisting of —(CH₂)ₘ—NH—C(═N—R₅)—NH₂, —R₆—NO₂, —R₆—J, and —R₆—C≡N;
R₄ is —CH(CH₂—R₇)—Q;
Q is selected from the group consisting of —CH—R₈, —C(═O)CH₃, —C(═O)CH₂Cl, —C(═O)CH₂Br, —C(═O)CH₂F, —C(═O)CHF₂, —C(═O)CF₃, —C(═O)C(═O)R₇, —C(═O)C(═O)NH—R₇, —C(═O)CO₂—R₇, —C(═O)CO₂H, —B(OH)₂, 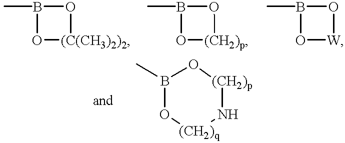
where p and q, independently, are 2 or 3;
W is cycloalkyl;
R₅ is selected from the group consisting of —NO₂, —C≡N, and —J;
R₆ is —(CH₂)ₘ—NH—C(═NH)—NH—;
R₇ is selected from the group consisting of phenyl, and alkyl having from one to eight carbons, said alkyl group being optionally substituted with one or more halogen atoms, aryl, or heteroaryl groups;
R₈ is selected from the group consisting of ═O, ═N—NHC(═O)—NH₂, ═N—OH, ═N—OCH₃, ═N—O—CH₂—C₆H₅, ═NNH—C(═S)—NH₂ and ═N—NH—J;
R₉ is selected from the group consisting of hydrogen and alkyl having from one to six carbons, said alkyl group being optionally substituted with one or more halogen atoms, aryl or heteroaryl groups;
J is a protecting group;
n is an integer from 3 to 10 ; and
m is an integer from 2 to 5.

69. The method of claim 68 wherein R₁ is —C≡N, —C(═O)OCH₃, phthalimido or —NH—SO₂CF₃.

70. The method of claim 68 wherein R₂ is H or cyclopentyl.

71. The method of claim 68 wherein R₃ is —(CH₂)₃—NH—C(═N—R₅)—NH₂.

72. The method of claim 68 wherein Q is —CH—R₈, —B(OH)₂, —C(═O)C(═O)NH—R₇, or has the structure: 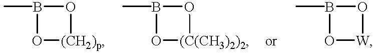
where W is pinane.

73. The method of claim 68 wherein R₅ is —NO₂, —C═N, —PMC, —MTR, —MTS, or Tos.

74. The method of claim 68 wherein R₇ is —CH(CH₃)₂, —(CH₂)₂—CH₃, —CH₂—CH₃, or —C₆H₅.

75. The method of claim 68 wherein R₈ is ═O, ═N—OH, ═N—O—CH₂—C₆H₅, ═NNH—C(═O)—NH₂ or ═NNH—C(═S)—NH₂.

76. The method of claim 68 wherein R₁ is —C(═O)OCH₃, phthalimido or —NH—SO₂CF₃; R₂ is cyclopentyl; R₃ is —(CH₂)₃—NH—C(═N—NO₂)—NH₂; R₇ is —CH(CH₃)₂; and R₈ is ═O.

77. The method of claim 68 wherein R₁ is —C≡N; R₂ is cyclopentyl; R₃ is —(CH₂)₃—NH—C(═N—NO₂)—NH₂ or —(CH₂)₃—NH—C(═N—J)—NH₂; R₇ is —CH(CH₃)₂; and R₈ is ═O.

78. The method of claim 68 wherein R₁ is C≡N; R₂ is cyclopentyl; R₃ is —(CH₂)₃—NH—C(═N—NO₂)—NH₂ or —(CH₂)₃—NH—C(═N—J)—NH₂; R₇ is —CH(CH₃)₂; Q is —CH—R₈; and R₈ is ═N—NHC(═O)—NH₂, ═N—OH, ═N—OCH₃, or ═N—O—CH₂—C₆H₅.

79. The method of claim 68 wherein R₁, R₂, R₃ and R₄ are selected from the group of substituents shown for the compounds in Table 1.

80. The method of claim 68 wherein said compound is selected from the group consisting of compounds A-J shown in Table 1.

81. The method of claim 68 wherein said tumor is a solid tumor.

82. The method of any one of claims 68-81 wherein said compound is administered to a mammal.

83. The method of claim 82 wherein said mammal is a human.

84. The method of claim 83 wherein said transformed cells are breast cancer cells, prostate cancer cells, tongue cancer cells, brain cancer cells, lung cancer cells, pancreatic cancer cells, ovarian cancer cells, or skin cancer cells.

85. The method of claim 84 wherein said transformed cells overproduce Bcl2 protein, and/or lack p53 protein.
